SEQUENCE LISTING (6 sequences)

SEQ ID NO: 1 - protein, 1353 aa, Human coronavirus OC43
MFLILLISLPTAFAVIGDLKCTSDNINDKDTGPPPISTDTVDVTNGLGTYYVLDRVYLNTTLFLNGYYPTSGSTYRNMALKGSVLLSRLWFKPPFLSDFINGIFAKVKNTKVIKDRVMYSEFPAITIGSTFVNTSYSVVVQPRTINSTQDGDNKLQGLLEVSVCQYNMCEYPQTICHPNLGNHRKELWHLDTGVVSCLYKRNFTYDVNADYLYFHFYQEGGTFYAYFTDTGVVTKFLFNVYLGMALSHYYVMPLTCNSKLTLEYWVTPLTSRQYLLAFNQDGIIFNAVDCMSDFMSEIKCKTQSIAPPTGVYELNGYTVQPIADVYRRKPNLPNCNIEAWLNDKSVPSPLNWERKTFSNCNFNMSSLMSFIQADSFTCNNIDAAKIYGMCFSSITIDKFAIPNGRKVDLQLGNLGYLQSFNYRIDTTATSCQLYYNLPAANVSVSRFNPSTWNKRFGFIEDSVFKPRPAGVLTNHDVVYAQHCFKAPKNFCPCKLNGSCVGSGPGKNNGIGTCPAGTNYLTCDNLCTPDPITFTGTYKCPQTKSLVGIGEHCSGLAVKSDYCGGNSCTCRPQAFLGWSADSCLQGDKCNIFANFILHDVNSGLTCSTDLQKANTDIILGVCVNYDLYGILGQGIFVEVNATYYNSWQNLLYDSNGNLYGFRDYITNRTFMIRSCYSGRVSAAFHANSSEPALLFRNIKCNYVFNNSLTRQLQPINYFDSYLGCVVNAYNSTAISVQTCDLTVGSGYCVDYSKNRRSRGAITTGYRFTNFEPFTVNSVNDSLEPVGGLYEIQIPSEFTIGNMVEFIQTSSPKVTIDCAAFVCGDYAACKSQLVEYGSFCDNINAILTEVNELLDTTQLQVANSLMNGVTLSTKLKDGVNFNVDDINFSPVLGCLGSECSKASSRSAIEDLLFDKVKLSDVGFVEAYNNCTGGAEIRDLICVQSYKGIKVLPPLLSENQISGYTLAATSASLFPPWTAAAGVPFYLNVQYRINGLGVTMDVLSQNQKLIANAFNNALYAIQEGFDATNSALVKIQAVVNANAEALNNLLQQLSNRFGAISASLQEILSRLDALEAEAQIDRLINGRLTALNAYVSQQLSDSTLVKFSAAQAMEKVNECVKSQSSRINFCGNGNHIISLVQNAPYGLYFIHFSYVPTKYVTARVSPGLCIAGDRGIAPKSGYFVNVNNTWMYTGSGYYYPEPITENNVVVMSTCAVNYTKAPYVMLNTSIPNLPDFKEELDQWFKNQTSVAPDLSLDYINVTFLDLQVEMNRLQEAIKVLNQSYINLKDIGTYEYYVKWPWYVWLLICLAGVAMLVLLFFICCCTGCGTSCFKKCGGCCDDYTGYQELVIKTSHDD

SEQ ID NO: 2 - protein, 1359 aa, Human coronavirus OC43
MFLILLISLPTAFAVIGDLKCPLDSRTGSLNNVDTGPPSISTATVDVTNGLGTYYVLDRVYLNTTLFLNGYYPTSGSTYRNMALKGTDKLSTLWFKPPFLSDFINGIFAKVKNTKVFKEGVMYSEFPAITIGSTFVNTSYSVVVQPRTINYGVNKLQGLLEVSVCQYNMCEYPQTICHPKLGNHFKELWHMDTGVVSCLYKRNFTYDVNATYLYFHFYQEGGTFYAYFTDTGVVTKFLFNVYLGMALSHYYVMPLTCISRRNIGFTLEYWVTPLTSRQYLLAFNQDGIIFNAVDCMSDFMSEIKCKTQSIAPPTGVYELNGYTVQPIADVYRRKPDLPNCNIEAWLNDKSVPSPLNWERKTFSNCNFNMSSLMSFIQVDSFTCNNIDAAKIYGMCFSSITIDKFAIPNGRKVDLQLGNLGYLQSFNYRIDTTATSCQLYYNLPAANVSVSRFNPSTWNKRFGFIENSVFKPQPAGVFTNHDVVYAQHCFKAPKNFCPCKLNSSLCVGSGPGKNNGIGTCPAGTNYLTCHNLCNPDPITFTGPYKCPQTKSLVGIGEHCSGLAVKSDYCGGNPCTCQPQAFLGWSADSCLQGDKCNIFANLILHDVNSGLTCSTDLQKANTDIKLGVCVNYDLYGISGQGIFVEVNATYYNSWQNLLYDSNGNLYGFRDYITNRTFMIRSCYSGRVSAAFHANSSEPALLFRNIKCNYVFNNSLIRQLQPINYFDSYLGCVVNAYNSTAISVQTCDLTVGSGYCVDYSKTRRSRRAITTGYRFTNFEPFTVNSVNDSLEPVGGLYEIQIPSEFTIGNMEEFIQTSSPKVTIDCAAFVCGDYAACKSQLVEYGSFCDNINAILTEVNELLDTTQLQVANSLMNGVTLSTKLKDGVNFNVDDINFSSVLGCLGSECSKASSRSAIEDLLFDKVKLSDVGFVAAYNNCTGGAEIRDLICVQSYKGIKVLPPLLSENQISGYTLAATSASLFPPWTAAAGVPFYLNVQYRINGLGVTMDVLSQNQKLIANAFNNALDAIQEGFDATNSALVKIQAVVNANAEALNNLLQQLSNRFGAISSSLQEILSRLDALEAEAQIDRLINGRLTALNAYVSQQLSDSTLVKFSAAQAMEKVNECVKSQSSRINFCGNGNHIISLVQNAPYGLYFIHFSYVPTKYVTAKVSTGLCIAGDRGIAPKSGYFVNVNNTWMYTGSGYYYPEPITENNVVVMSTCAVNYTKAPYVMLNTSTSNLPDFREELDQWFKNQTSVAPDLSLDYINVTLLDLQVEMNRLQEAIKVLNQSYINLKDIGTYEYYVKWPWYVWLLIGLAGVAMLVLLFFICCCTGCGTSCFKKCGGCCDDYTGYQELVIKTSHDD

SEQ ID NO: 3 - protein, 1356 aa, Human coronavirus HKU1
MLLIIFILPTTLAVIGDFNCTNFAINDLNTTVPRISEYVVDVSYGLGTYYILDRVYLNTTILFTGYFPKSGANFRDLSLKGTTYLSTLWYQKPFLSDFNNGIFSRVKNTKLYVNKTLYSEFSTIVIGSVFINNSYTIVVQPHNGVLEITACQYTMCEYPHTICKSKGSSRNESWHFDKSEPLCLFKKNFTYNVSTDWLYFHFYQERGTFYAYYADSGMPTTFLFSLYLGTLLSHYYVLPLTCNAISSNTDNETLQYWVTPLSKRQYLLKFDNRGVITNAVDCSSSFFSEIQCKTKSLLPNTGVYDLSGFTVKPVATVHRRIPDLPDCDIDKWLNNFNVPSPLNWERKIFSNCNFNLSTLLRLVHTDSFSCNNFDESKIYGSCFKSIVLDKFAIPNSRRSDLQLGSSGFLQSSNYKIDTTSSSCQLYYSLPAINVTINNYNPSSWNRRYGFNNFNLSSHSVVYSRYCFSVNNTFCPCAKPSFASSCKSHKPPSASCPIGTNYRSCESTTVLDHTDWCRCSCLPDPITAYDPRSCSQKKSLVGVGEHCAGFGVDEEKCGVLDGSYNVSCLCSTDAFLGWSYDTCVSNNRCNIFSNFILNGINSGTTCSNDLLQPNTEVFTDVCVDYDLYGITGQGIFKEVSAVYYNSWQNLLYDSNGNIIGFKDFVTNKTYNIFPCYAGRVSAAFHQNASSLALLYRNLKCSYVLNNISLTTQPYFDSYLGCVFNADNLTDYSVSSCALRMGSGFCVDYNSPSSSSSRRKRRSISASYRFVTFEPFNVSFVNDSIESVGGLYEIKIPTNFTIVGQEEFIQTNSPKVTIDCSLFVCSNYAACHDLLSEYGTFCDNINSILDEVNGLLDTTQLHVADTLMQGVTLSSNLNTNLHFDVDNINFKSLVGCLGPHCGSSSRSFFEDLLFDKVKLSDVGFVEAYNNCTGGSEIRDLLCVQSFNGIKVLPPILSESQISGYTTAATVAAMFPPWSAAAGIPFSLNVQYRINGLGVTMDVLNKNQKLIATAFNNALLSIQNGFSATNSALAKIQSVVNSNAQALNSLLQQLFNKFGAISSSLQEILSRLDALEAQVQIDRLINGRLTALNAYVSQQLSDISLVKFGAALAMEKVNECVKSQSPRINFCGNGNHILSLVQNAPYGLLFMHFSYKPISFKTVLVSPGLCISGDVGIAPKQGYFIKHNDHWMFTGSSYYYPEPISDKNVVFMNTCSVNFTKAPLVYLNHSVPKLSDFESELSHWFKNQTSIAPNLTLNLHTINATFLDLYYEMNLIQESIKSLNNSYINLKDIGTYEMYVKWPWYVWLLISFSFIIFLVLLFFICCCTGCGSACFSKCHNCCDEYGGHHDFVIKTSHDD

SEQ ID NO: 4 - protein, 1351 aa, Human coronavirus HKU1
MFLIIFILPTTLAVIGDFNCTNSFINDYNKTIPRISEDVVDVSLGLGTYYVLNRVYLNTTLLFTGYFPKSGANFRDLALKGSKYLSTLWYKPPFLSDFNNGIFSKVKNTKLYVNNTLYSEFSTIVIGSVFVNTSYTIVVQPHNGILEITACQYTMCEYPHTVCKSKGSIRNESWHIDSSEPLCLFKKNFTYNVSADWLYFHFYQERGVFYAYYADVGMPTTFLFSLYLGTILSHYYVMPLTCKAISSNTDNETLEYWVTPLSRRQYLLNFDEHGVITNAVDCSSSFLSEIQCKTQSFAPNTGVYDLSGFTVKPVATVYRRIPNLPDCDIDNWLNNVSVPSPLNWERRIFSNCNFNLSTLLRLVHVDSFSCNNLDKSKIFGSCFNSITVDKFAIPNRRRDDLQLGSSGFLQSSNYKIDISSSSCQLYYSLPLVNVTINNFNPSSWNRRYGFGSFNVSSYDVVYSDHCFSVNSDFCPCADPSVVNSCVKSKPLSAICPAGTKYRHCDLDTTLYVNNWCRCSCLPDPISTYSPNTCPQKKVVVGIGEHCPGLGINEEKCGTQLNHSSCSCSPDAFLGWSFDSCISNNRCNIFSNFIFNGINSGTTCSNDLLYSNTEVSTGVCVNYDLYGITGQGIFKEVSAAYYNNWQNLLYDSNGNIIGFKDFLTNKTYTILPCYSGRVSAAFYQNSSSPALLYRNLKCSYVLNNISFISQPFYFDSYLGCVLNAVNLTSYSVSSCDLRMGSGFCIDYALPSSRRKRRGISSPYRFVTFEPFNVSFVNDSVETVGGLFEIQIPTNFTIAGHEEFIQTSSPKVTIDCSAFVCSNYAACHDLLSEYGTFCDNINSILNEVNDLLDITQLQVANALMQGVTLSSNLNTNLHSDVDNIDFKSLLGCLGSQCGSSSRSLLEDLLFNKVKLSDVGFVEAYNNCTGGSEIRDLLCVQSFNGIKVLPPILSETQISGYTTAATVAAMFPPWSAAAGVPFSLNVQYRINGLGVTMDVLNKNQKLIANAFNKALLSIQNGFTATNSALAKIQSVVNANAQALNSLLQQLFNKFGAISSSLQEILSRLDNLEAQVQIDRLINGRLTALNAYVSQQLSDITLIKAGASRAIEKVNECVKSQSPRINFCGNGNHILSLVQNAPYGLLFIHFSYKPTSFKTVLVSPGLCLSGDRGIAPKQGYFIKQNDSWMFTGSSYYYPEPISDKNVVFMNSCSVNFTKAPFIYLNNSIPNLSDFEAEFSLWFKNHTSIAPNLTFNSHINATFLDLYYEMNVIQESIKSLNSSFINLKEIGTYEMYVKWPWYIWLLIVILFIIFLMILFFICCCTGCGSACFSKCHNCCDEYGGHNDFVIKASHDD

SEQ ID NO: 5 - protein, 1173 aa, Human coronavirus 229E
MFVLLVAYALLHIAGCQTTNGLNTSYSVCNGCVGYSENVFAVESGGYIPSDFAFNNWFLLTNTSSVVDGVVRSFQPLLLNCLWSVSGLRFTTGFVYFNGTGRGDCKGFSSDVLSDVIRYNLNFEENLRRGTILFKTSYGVVVFYCTNNTLVSGDAHIPFGTVLGNFYCFVNTTIGNETTSAFVGALPKTVREFVISRTGHFYINGYRYFTLGNVEAVNFNVTTAETTDFCTVALASYADVLVNVSQTSIANIIYCNSVINRLRCDQLSFDVPDGFYSTSPIQSVELPVSIVSLPVYHKHTFIVLYVDFKPQSGGGKCFNCYPAGVNITLANFNETKGPLCVDTSHFTTKYVAVYANVGRWSASINTGNCPFSFGKVNNFVKFGSVCFSLKDIPGGCAMPIVANWAYSKYYTIGSLYVSWSDGDGITGVPQPVEGVSSFMNVTLDKCTKYNIYDVSGVGVIRVSNDTFLNGITYTSTSGNLLGFKDVTKGTIYSITPCNPPDQLVVYQQAVVGAMLSENFTSYGFSNVVELPKFFYASNGTYNCTDAVLTYSSFGVCADGSIIAVQPRNVSYDSVSAIVTANLSIPSNWTTSVQVEYLQITSTPIVVDCSTYVCNGNVRCVELLKQYTSACKTIEDALRNSARLESADVSEMLTFDKKAFTLANVSSFGDYNLSSVIPSLPTSGSRVAGRSAIEDILFSKLVTSGLGTVDADYKKCTKGLSIADLACAQYYNGIMVLPGVADAERMAMYTGSLIGGIALGGLTSAVSIPFSLAIQARLNYVALQTDVLQENQKILAASFNKAMTNIVDAFTGVNDAITQTSQALQTVATALNKIQDVVNQQGNSLNHLTSQLRQNFQAISSSIQAIYDRLDTIQADQQVDRLITGRLAALNVFVSHTLTKYTEVRASRQLAQQKVNECVKSQSKRYGFCGNGTHIFSIVNAAPEGLVFLHTVLLPTQYKDVEAWSGLCVDGTNGYVLRQPNLALYKEGNYYRITSRIMFEPRIPTMADFVQIENCNVTFVNISRSELQTIVPEYIDVNKTLQELSYKLPNYTVPDLVVEQYNQTILNLTSEISTLENKSAELNYTVQKLQTLIDNINSTLVDLKWLNRVETYIKWPWWVWLCISVVLIFVVSMLLLCCCSTGCCGFFSCFASSIRGCCESTKLPYYDVEKIHIQ

SEQ ID NO: 6 - protein, 1356 aa, Human coronavirus NL63
MKLFLILLVLPLASCFFTCNSNANLSMLQLGVPDNSSTIVTGLLPTHWFCANQSTSVYSANGFFYIDVGNHRSAFALHTGYYDANQYYIYVTNEIGLNASVTLKICKFSRNTTFDFLSNASSSFDCIVNLLFTEQLGAPLGITISGETVRLHLYNVTRTFYVPAAYKLTKLSVKCYFNYSCVFSVVNATVTVNVTTHNGRVVNYTVCDDCNGYTDNIFSVQQDGRIPNGFPFNNWFLLTNGSTLVDGVSRLYQPLRLTCLWPVPGLKSSTGFVYFNATGSDVNCNGYQHNSVVDVMRYNLNFSANSLDNLKSGVIVFKTLQYDVLFYCSNSSSGVLDTTIPFGPSSQPYYCFINSTINTTHVSTFVGILPPTVREIVVARTGQFYINGFKYFDLGFIEAVNFNVTTASATDFWTVAFATFVDVLVNVSATNIQNLLYCDSPFEKLQCEHLQFGLQDGFYSANFLDDNVLPETYVALPIYYQHTDINFTATASFGGSCYVCKPHQVNISLNGNTSVCVRTSHFSIRYIYNRVKSGSPGDSSWHIYLKSGTCPFSFSKLNNFQKFKTICFSTVEVPGSCNFPLEATWHYTSYTIVGALYVTWSEGNSITGVPYPVSGIREFSNLVLNNCTKYNIYDYVGTGIIRSSNQSLAGGITYVSNSGNLLGFKNVSTGNIFIVTPCNQPDQVAVYQQSIIGAMTAVNESRYGLQNLLQLPNFYYVSNGGNNCTTAVMTYSNFGICADGSLIPVRPRNSSDNGISAIITANLSIPSNWTTSVQVEYLQITSTPIVVDCATYVCNGNPRCKNLLKQYTSACKTIEDALRLSAHLETNDVSSMLTFDSNAFSLANVTSFGDYNLSSVLPQRNIRSSRIAGRSALEDLLFSKVVTSGLGTVDVDYKSCTKGLSIADLACAQYYNGIMVLPGVADAERMAMYTGSLIGGMVLGGLTSAAAIPFSLALQARLNYVALQTDVLQENQKILAASFNKAINNIVASFSSVNDAITQTAEAIHTVTIALNKIQDVVNQQGSALNHLTSQLRHNFQAISNSIQAIYDRLDSIQADQQVDRLITGRLAALNAFVSQVLNKYTEVRGSRRLAQQKINECVKSQSNRYGFCGNGTHIFSIVNSAPDGLLFLHTVLLPTDYKNVKAWSGICVDGIYGYVLRQPNLVLYSDNGVFRVTSRVMFQPRLPVLSDFVQIYNCNVTFVNISRVELHTVIPDYVDVNKTLQEFAQNLPKYVKPNFDLTPFNLTYLNLSSELKQLEAKTASLFQTTVELQGLIDQINSTYVDLKLLNRFENYIKWPWWVWLIISVVFVVLLSLLVFCCLSTGCCGCCNCLTSSMRGCCDCGSTKLPYYEFEKVHVQ

(19) United States
(12) Patent Application Publication
(10) Pub. No.: US 2025/0093335 A1
(43) Pub. Date: Mar. 20, 2025

(54) METHOD FOR EVALUATING SEVERITY RISK OF CORONAVIRUS INFECTIOUS DISEASE, AND KIT FOR EVALUATING SEVERITY RISK OF CORONAVIRUS INFECTION DISEASE

(71) Applicant: TOHOKU UNIVERSITY, Sendai-shi, Miyagi (JP)

(72) Inventor: Tsutomu TAMADA, Sendai-shi, Miyagi (JP)

(21) Appl. No.: 18/291,704

(22) PCT Filed: Mar. 8, 2022
(86) PCT No.: PCT/JP2022/009911

§ 371 Date: Jan. 24, 2024

(30) Foreign Application Priority Data
Jul. 29, 2021 (JP) 2021-124390

(51) Int. Cl. G01N 33/50

(52) U.S. Cl. CPC G01N 33/5091; G01N 33/505; G01N 2333/165

(57) ABSTRACT

Provided is a method for evaluating a severity risk of a coronavirus infectious disease. The method includes: (a) a step of bringing a subject-derived T cell into contact with a protein or fragment thereof derived from another kind of coronavirus which is a different kind of coronavirus from a causal virus of the coronavirus infectious disease; and (b) a step of evaluating a severity risk of the coronavirus infectious disease based on responsiveness of the T cell to the protein or fragment thereof. Also provided is a kit for evaluating a severity risk of a coronavirus infectious disease including a protein or fragment thereof derived from another kind of coronavirus which is a different kind of coronavirus from the causal virus of the coronavirus infectious disease.

### TECHNICAL FIELD

The present invention relates to a method for evaluating a severity risk of a coronavirus infectious disease, and a kit for evaluating a severity risk of a coronavirus infectious disease. The present application claims priority from the Japanese Patent Application No. 2021-124390, filed on Jul. 29, 2021, the content of which is incorporated herein by reference.

### BACKGROUND ART

COVID-19 is a virus infection of which the causal virus is SARS-CoV-2. Among COVID-19 cases, severe cases are up to about 20% (moderate to severe cases are 14%, critical cases are 5%, and death cases are 2%). Cases of elderly people and cases with underlying diseases such as high-blood pressure, diabetic, cardiovascular disease, chronic respiratory disease, and cancer have a high severity risk, and it is considered that approximately from 6 to 14% of those cases get severe. In COVID-19 epidemic areas, severe patients, elderly people, and patients with underlying diseases occupy ICUs and hospital beds, respectively, leading to healthcare system collapse.

It has been reported that SARS-CoV-2-specific CD8 positive T cells and CD4 positive T cells were detected in 70% and 100% of COVID-19 convalescent patients, respectively (Non-Patent Literature 1). Non-Patent Literature 1 also reports that CD4 positive T cells reacting with SARS-CoV-2 were detected in about 40 to 60% of non-infected individuals.

### CITATION LIST

### Non-Patent Literature

Non-Patent Literature 1: Alba Grifoni et al., Targets of T Cell Responses to SARS-CoV-2 Coronavirus in Humans with COVID-19 Disease and Unexposed Individuals. Cell. 2020 Jun. 25; 181(7):1489-1501.e15.

### SUMMARY OF INVENTION

### Technical Problem

If it is possible to distinguish in advance between an individual who is likely to have severe COVID-19 and an individual who is unlikely to have severe COVID-19, active and efficient medical and governmental intervention can be performed. For example, there is a possibility that severe cases can be suppressed by determining a severity risk at an early stage of infection and performing active therapeutic intervention. In addition, after the spread of vaccination as well, periodical confirmation of the maintenance of immunocompetence can serve as an index for balancing medical care and economy. At present, the severity risk is only judged by age and the presence or absence of underlying diseases, and there is no test method that can evaluate the severity risk of COVID-19. Thus, a test method capable of easily determining the severity risk of COVID-19 is required to be developed.

Even in a case where the spread of vaccination overcomes the epidemic of COVID-19, a new coronavirus infectious disease may spread in the future. For such a new coronavirus infectious disease as well, if it is possible to distinguish in advance between an individual who is likely to become severe and an individual who is unlikely to become severe, efficient medical and governmental intervention becomes possible.

Accordingly, the present invention is directed to providing a method for evaluating a severity risk of a coronavirus infectious disease and a kit for evaluating a severity risk of a coronavirus infectious disease, which can easily determine the severity risk of the coronavirus infectious disease.

### Solution to Problem

The present invention includes the following aspects.

[1] A method for evaluating a severity risk of a coronavirus infectious disease, the method including: (a) a step of bringing a subject-derived T cell into contact with a protein or fragment thereof derived from another kind of coronavirus which is a different kind of coronavirus from a causal virus of the coronavirus infectious disease; and (b) a step of evaluating a severity risk of the coronavirus infectious disease based on responsiveness of the T cell to the protein or fragment thereof.

[2] The method for evaluating a severity risk of a coronavirus infectious disease according to [1], in which the protein or fragment thereof is derived from a first of another kind of coronavirus utilizing a different molecule as a receptor from a molecule utilized as a receptor by the causal virus, and the step (b) is a step of evaluating that the severity risk of the coronavirus infectious disease is high in a case where the responsiveness of the T cell to the protein or fragment thereof is low.

[3] The method for evaluating a severity risk of a coronavirus infectious disease according to [1], in which the protein or fragment thereof is derived from a second of another kind of coronavirus utilizing as a receptor the same molecule as the molecule utilized as a receptor by the causal virus, and the step (b) is a step of evaluating that the severity risk of the coronavirus infectious disease is high in a case where the responsiveness of the T cell to the protein or fragment thereof is high.

[4] The method for evaluating a severity risk of a coronavirus infectious disease according to [1], in which the step (a) includes: (a1) a step of bringing the T cell into contact with a protein or fragment thereof derived from a first of another kind of coronavirus utilizing a different molecule as a receptor from a molecule utilized as a receptor by the causal virus; and (a2) a step of bringing the T cell into contact with a protein or fragment thereof derived from a second of another kind of coronavirus utilizing as a receptor the same molecule as the molecule utilized as a receptor by the causal virus, and the step (b) includes: (b1) a step of measuring a first responsiveness of the T cell to the protein or fragment thereof derived from the first of another kind of coronavirus; (b2) a step of measuring a second responsiveness of the T cell to the protein or fragment thereof derived from the second of another kind of coronavirus; and (b3) a step of evaluating a severity risk of the coronavirus infectious disease based on a ratio value of the second responsiveness to the first responsiveness (second responsiveness/first responsiveness).

[5] The method for evaluating a severity risk of a coronavirus infectious disease according to any one of [1] to [4], in which the coronavirus from which the causal virus and the protein or fragment thereof are derived are of the genus Betacoronavirus or the genus Alphacoronavirus.

[6] The method for evaluating a severity risk of a coronavirus infectious disease according to any one of [1] to [5], in which the causal virus is SARS-CoV-2.

[7] A kit for evaluating a severity risk of a coronavirus infectious disease, the kit including a protein or fragment thereof derived from another kind of coronavirus which is a different kind of coronavirus from a causal virus of the coronavirus infectious disease.

[8] The kit for evaluating a severity risk of a coronavirus infectious disease according to [7], the kit further including a specific binding substance for a cytokine secreted by a T cell in response to antigen stimulation.

[9] The kit for evaluating a severity risk of a coronavirus infectious disease according to [8], the kit including: the specific binding substance bound to a solid-phase carrier; and the specific binding substance labeled with a labeling substance.

[10] The kit for evaluating a severity risk of a coronavirus infectious disease according to [8] or [9], in which the cytokine is at least one selected from the group consisting of interferon γ, interleukin 2, and tumor-necrosis factor alpha.

[11] The kit for evaluating a severity risk of a coronavirus infectious disease according to any one of [7] to [10], in which the protein or fragment thereof includes a protein or fragment thereof derived from a first of another kind of coronavirus utilizing a different molecule as a receptor from a molecule utilized as a receptor by the causal virus, and a protein or fragment thereof derived from a second of another kind of coronavirus utilizing as a receptor the same molecule as the molecule utilized as a receptor by the causal virus.

[12] The kit for evaluating a severity risk of a coronavirus infectious disease according to any one of [7] to [11], in which the causal virus and the coronavirus from which the protein or fragment thereof is derived are of the genus Betacoronavirus or the genus Alphacoronavirus.

[13] The kit for evaluating a severity risk of a coronavirus infectious disease according to any one of [7] to [12], in which the causal virus is SARS-CoV-2.

### Advantageous Effects of Invention

According to the present invention, provided are a method for evaluating a severity risk of a coronavirus infectious disease and a kit for evaluating a severity risk of a coronavirus infectious disease, which can easily determine a severity risk of a coronavirus infectious disease.

### BRIEF DESCRIPTION OF DRAWINGS

FIG. 1 illustrates results of an ELISPOT assay using PBMCs isolated from blood of COVID-19 convalescent patients and healthy subjects.

FIG. 2A illustrates a graph quantifying the results of the ELISPOT assay using HKU1-S protein in FIG. 1.

FIG. 2B illustrates a graph quantifying the results of the ELISPOT assay using OC43-S protein in FIG. 1.

FIG. 2C illustrates a graph quantifying the results of the ELISPOT assay using SARS-CoV-2-S protein in FIG. 1.

FIG. 3A illustrates results of the ELISPOT assay using S protein of HCoV-HKU1 (HKU1-S) as a T-lymphocyte stimulating antigen.

FIG. 3B illustrates results of the ELISPOT assay using S protein of HCoV-OC43 (OC43-S) as the T-lymphocyte stimulating antigen.

FIG. 4A illustrates results of the ELISPOT assay using S protein of HCoV-NL63 (NL63-S) as the T-lymphocyte stimulating antigen.

FIG. 4B illustrates results of the ELISPOT assay using S protein of HCoV-229E (229E-S) as the T-lymphocyte stimulating antigen.

FIG. 5A illustrates results of the ELISPOT assay using S protein of SARS-CoV-2 (CoV2-S) as the T-lymphocyte stimulating antigen.

FIG. 5B illustrates results of the ELISPOT assay using SARS-CoV-2 peptide pool (CoV2-all) as the T-lymphocyte stimulating antigen.

FIG. 6A is a graph collectively illustrating the results of Moderate I, Moderate II, and Severe in FIG. 3A as [Moderate or severer].

FIG. 6B is a graph collectively illustrating the results of Moderate I, Moderate II, and Severe in FIG. 3B as [Moderate or severer].

FIG. 7A is a graph collectively illustrating the results of Moderate I, Moderate II, and Severe in FIG. 4A as [Moderate or severer].

FIG. 7B is a graph collectively illustrating the results of Moderate I, Moderate II, and Severe in FIG. 4B as [Moderate or severer].

FIG. 8A is a graph collectively illustrating the results of Moderate I, Moderate II, and Severe in FIG. 5A as [Moderate or severer].

FIG. 8B is a graph collectively illustrating the results of Moderate I, Moderate II, and Severe in FIG. 5B as [Moderate or severer].

FIG. 9 is a graph illustrating severity risk values (Y) calculated by an equation (1) described below, divided into healthy subjects, mild cases, and moderate or severer cases.

FIG. 10A illustrates results of the ELISPOT assay using HKU1-S as the T-lymphocyte stimulating antigen before (Pre) and after (Post) vaccination.

FIG. 10B illustrates results of the ELISPOT assay using OC43-S as the T-lymphocyte stimulating antigen before (Pre) and after (Post) vaccination.

FIG. 11A illustrates results of the ELISPOT assay using HCoV-NL63 as the T-lymphocyte stimulating antigen before (Pre) and after (Post) vaccination.

FIG. 11B illustrates results of the ELISPOT assay using 229E-S as the T-lymphocyte stimulating antigen before (Pre) and after (Post) vaccination.

FIG. 12A illustrates results of the ELISPOT assay using CoV2-S as the T-lymphocyte stimulating antigen before (Pre) and after (Post) vaccination.

FIG. 12B illustrates results of the ELISPOT assay using CoV2-all as the T-lymphocyte stimulating antigen before (Pre) and after (Post) vaccination.

FIG. 13A is a scatter diagram illustrating severity risk values (Y) before (Pre) and after (Post) vaccination.

FIG. 13B illustrates changes in the severity risk values (Y) before (Pre) and after (Post) vaccination.

FIG. 14A illustrates a result of analysis of a correlation between the severity risk values (Y) and an age.

FIG. 14B illustrates a result of analysis of a correlation between the severity risk values (Y) and a BMI.

FIG. 15A illustrates a result of analysis of a correlation between the severity risk values (Y) and a smoking index BI.

FIG. 15B illustrates a result of analysis of a correlation between the severity risk values (Y) and a smoking history.

FIG. 16A illustrates a result of analysis of a correlation between severity and an age in subjects of Example 2.

FIG. 16B illustrates a result of analysis of a correlation between severity and a BMI in the subjects of Example 2.

FIG. 17A illustrates a result of analysis of a correlation between severity and a smoking index BI in the subjects of Example 2.

FIG. 17B illustrates a result of analysis of a correlation between severity and a smoking history in the subjects of Example 2.

### DESCRIPTION OF EMBODIMENTS

### Definitions

The term “comprise” means that an element other than the element in question may be included. The term “consist of” means that an element other than the element in question is not included. The term “consist essentially of” means that an element other than the element in question is not included in an aspect in which a special function is exhibited (such as an aspect in which the effect of the invention is completely lost). As used herein, references to “comprise” encompass an aspect of “consist of” and an aspect of “consist essentially of”.

A protein, a peptide, and a cell may be isolated. The term “isolated” means a natural state or a state of being separated from other components. An “isolated” one may be substantially free of other components. The phrase “substantially free of other components” means that the content of the other components contained in an isolated component is negligible. The content of the other components contained in the isolated component can be, for example, 10 mass % or less, 5 mass % or less, 4 mass % or less, 3 mass % or less, 2 mass % or less, 1 mass % or less, 0.5 mass % or less, or 0.1 mass % or less. The proteins, peptides, and cells described herein can be isolated proteins, isolated peptides, and isolated cells.

The term “coronavirus” refers to a virus belonging to the family Coronaviridae. The viruses belonging to the family Coronaviridae are single-stranded positive-stranded RNA viruses and each have an envelope. Classification of viruses is, for example, in accordance with the classification of the International Committee on Taxonomy of Viruses (ICTV). The coronavirus is classified in the genus Nidovirales of the family Coronaviridae on the virus taxonomy. The family Coronaviridae is further divided into the subfamily Coronavirinae (Orthocoronavirinae) and the subfamily Letovirinae. The subfamily Coronavirinae is further divided into four genera: Alpha(α)coronavirus, Beta(β)coronavirus, Gamma(γ)coronavirus, and Delta(δ)coronavirus. SARS-CoV-1, MERS-CoV, and SARS-CoV-2 are classified in the genus Betacoronavirus. Among four human cold coronaviruses, HCoV-HKU1 and HCoV-OC43 are classified in the genus Betacoronavirus. The remaining human cold coronaviruses, HCoV-229E and HCoV-NL63, are classified in the genus Alphacoronavirus (El-Sayed A, et al. Environ Sci Pollut Res Int. 2021 April; 28 (16): 19589-19600). In comparison of amino acid sequence homology, SARS-CoV-2 has a high homology to HCoV-HKU1 (75.7%) and HCoV-OC43 (74.8%), which belong to the same genus Betacoronavirus. In contrast, SARS-CoV-2 has a low homology to HCoV-229E (64.9%) and HCoV-NL63 (56.3%), which belong to the genus Alphacoronavirus (Cueno M E, et al. Front Med (Lausanne). 2021 Jan. 14; 7: 594439.). When the homology is high, it can be expected that cross-reactivity becomes high cellular-immunologically.

In one embodiment, the coronavirus is Human coronavirus. The term “Human coronavirus” refers to coronavirus that infects humans. Examples of the Human coronavirus include: HCoV-229E and HCoV-NL63 of the genus Alphacoronavirus; and HCoV-HKU1, HCoV-OC43, SARS-CoV, MERS-CoV, and SARS-CoV-2 of the genus Betacoronavirus. SARS-CoV-2 encompasses existing variants such as alpha, beta, gamma, delta, lambda, mu, epsilon, eta, iota, kappa, and omicron strains, as well as variants that may occur in the future.

The term “coronavirus infectious disease” refers to a disease caused by infection with coronavirus. For example, HCoV-229E, HCoV-NL63, HCoV-HKU1, and HCoV-OC43 each cause a common cold. SARS-CoV causes a severe acute respiratory syndrome (SARS). MERS-CoV causes a Middle East respiratory syndrome (MERS). SARS-CoV-2 causes a new coronavirus infectious disease (COVID-19).

The term “severity risk of coronavirus infectious disease” refers to a risk of progression of a coronavirus infectious disease symptom. For example, in a case where a subject is a coronavirus-infected individual, the severity risk of the coronavirus infectious disease can be a qualitative index of a possibility that the symptom of the coronavirus will progress, leading to a situation in which active medical intervention is required. For example, in a case where a subject is a coronavirus-non-infected individual, the severity risk of the coronavirus infectious disease can be a qualitative index of a possibility that when the subject is infected with coronavirus, the symptom of the coronavirus will progress, leading to a situation in which active medical intervention is required. The severity risk of the coronavirus infectious disease may be, for example, a risk of developing various symptoms associated with progression of the infection in a case where a subject is infected with coronavirus. Examples of the symptoms developed in association with progression of the coronavirus infectious disease include dyspnea, pneumonitis, hypoxia, respiratory failure, acute respiratory distress syndrome, acute pulmonary disorder, septicemia, and multi-organ failure. In a case where the coronavirus infectious disease is COVID-19, the severity risk may be a risk of becoming a moderate or severer case (for example, Moderate I or severer, Moderate II or severer, or Severe or severer according to Clinical Management of Patients with COVID-19, A guide for front-line healthcare workers, Version 5 (Ministry of Health, Labour and Welfare) (https://www.mhlw.go.jp/content/000785119.pdf)). According to the Clinical Management of Patients with COVID-19, A guide for front-line healthcare workers, Version 5, the severity of COVID-19 is classified as follows.

Mild: No respiratory symptom with oxygen saturation (SpO²)≥96%, or coughing only without dyspnea. No finding of pneumonia is obtained in any case.

Moderate I (without respiratory failure): Dyspnea and pneumonia findings are obtained with 93%<SpO2<96%.

Moderate II (with respiratory failure): Oxygen administration is required with SpO2≤93%.

Severe: Admission to ICU or a mechanical ventilator is required.

In a case where a subject is a healed individual after suffering from COVID-19, the severity risk may be a risk that the subject will be infected with SARS-CoV-2 again in the future to develop COVID-19 and become severe due to emergence of a SARS-CoV-2 variant.

In a case where the subject is a healed individual after suffering from COVID-19, the severity risk may be a risk that the subject will be infected with SARS-CoV-2 again in the future to develop COVID-19 and become severe due to a decrease in immune memory of the subject.

In a case where a subject is a PCR positive asymptomatic pathogen carrier in the PCR test of SARS-CoV-2, the subject often goes through a course of either getting severe or being healed spontaneously about one week after the onset of COVID-19. In this case, the severity risk may be a risk of developing COVID-19 to become severe.

In a case where a subject is a SARS-CoV-2 vaccinee, the severity risk may be a risk that the subject will be infected with SARS-CoV-2 again in the future to develop COVID-19 and become severe due to emergence of a SARS-CoV-2 variant having a low vaccine sensitivity.

In a case where a subject is a SARS-CoV-2 vaccinee, the severity risk may be a risk that the subject will be infected with SARS-CoV-2 again in the future to develop COVID-19 and become severe, together with reduction in the vaccine effect in the subject.

The term “antibody” means immunoglobulin having an antigen-binding activity. The antibody is not limited to an intact antibody and may be an antigen-binding fragment as long as it has the antigen-binding activity. As used herein, the term “antibody” encompasses an antigen-binding fragment. The “antigen-binding fragment” is polypeptide including a portion of an antibody, which retains an antigen-binding property of the original antibody. The antigen-binding fragment preferably includes all six complementarity determining regions (CDRs) of the original antibody. That is, the antigen-binding fragment preferably includes all of CDR1, CDR2, and CDR3 of the heavy chain variable region and CDR1, CDR2, and CDR3 of the light chain variable region. Examples of the antigen-binding fragment include Fab, Fab′, F(ab′)₂, variable region fragment (Fv), disulfide-linked Fv, single chain Fv (scFv), and sc(Fv)₂.

The antibody may be derived from any organism. Examples of the organism from which the antibody is derived include, but are not limited to, mammals (humans, mice, rats, rabbits, horses, cows, pigs, monkeys, dogs, etc.), and birds (chickens, ostriches).

The antibody may be of any class or subclass of immunoglobulin. The antibody may be a monoclonal antibody or a polyclonal antibody, but is preferably a monoclonal antibody.

The antibody can be produced by a known method such as an immunization method, a hybridoma method, or a phage display method.

The term “binding pair” refers to two molecules (a pair of molecules) that bind to each other. The term “first element” of a binding pair refers to one molecule of the pair of molecules forming the binding pair. The term “second element” of a binding pair refers to the other molecule of the pair of molecules forming the binding pair. Examples of the binding pair include biotin or a derivative thereof and avidin or a derivative thereof (streptavidin, neutravidin, and the like).

The “receptor” refers to a molecule to which a virus binds upon entry into a cell. The receptor is usually present on a cell surface layer of a host cell subjected to virus infection. Coronavirus is thought to bind to a receptor present on a cell surface layer of a host cell with spike protein to infect the host cell. The receptors for major species of alphacoronavirus and betacoronavirus are indicated in Table 1. In Table 1, APN1 represents aminopeptidase N, ACE2 represents angiotensin converting enzyme 2, and DPP4 represents dipeptidyl peptidase-4.

TABLE 1

| Genus | Species | Infection | Receptor |
|---|---|---|---|
| Alphacoronavirus | HCoV-229E | Common cold | APN1 |
| | HCoV-NL63 | Common cold | ACE2 |
| Betacoronavirus | HCoV-OC43 | Common cold | O-acetyl |
| | | | sialic acid |
| | SARS-CoV | Severe acute respiratory | ACE2 |
| | | syndrome (SARS) | |
| | HCoV-HKU1 | Common cold, | O-acetyl |
| | | pneumonia | sialic acid |
| | MERS-CoV | Middle East respiratory | DPP4 |
| | | syndrome (MERS) | |
| | SARS-CoV-2 | COVID-19 | ACE2 |

HCoC-NL63 and SARS-CoV each use the same molecule as a molecule utilized by SARS-CoV-2 (that is, ACE2) as a receptor.

HCoC-229E, HCoC-OC43, HCoC-HKU1, and MERS-CoV each use a molecule different from a molecule utilized by SARS-CoV-2 as the receptor.

### [Method for Evaluating Severity Risk of Coronavirus Infectious Disease]

A first aspect of the present disclosure is a method for evaluating a severity risk of a coronavirus infectious disease. The method according to the present aspect includes the following steps (a) and (b):
(a) a step of bringing a subject-derived T cell into contact with a protein or fragment thereof derived from another kind of coronavirus which is a different kind of coronavirus from a causal virus of the coronavirus infectious disease; and
(b) a step of evaluating a severity risk of the coronavirus infectious disease based on responsiveness of the T cell to the protein or fragment thereof.

In one embodiment, the evaluation method according to the present aspect can be performed using an ELISPOT assay technique. As a T-cell stimulating antigen in the ELISPOT assay, it is possible to use a protein or fragment thereof derived from another kind of coronavirus which is a different kind of coronavirus from the coronavirus infectious disease that is an evaluation target of the severity risk. The evaluation method according to the present aspect is a method performed in vitro.

<Step (a)>

In the step (a), a subject-derived T cell is brought into contact with a protein or fragment thereof derived from another kind of coronavirus which is a different kind of coronavirus from the causal virus of the coronavirus infectious disease.

### (Subjects)

The term “subject” refers to an individual to be evaluated for severity risk of the coronavirus infectious disease. The subject is not particularly limited as long as it is an animal to suffer from the coronavirus infectious disease. Examples of the subject include humans and non-human mammals. Examples of the non-human mammals include, but are not limited to, primates such as monkeys, chimpanzees, gorillas, and marmosets, rodents such as mice, guinea pigs, hamsters, and rats, and carnivora such as dogs and cats. In one embodiment, the subject is a human.

The subject may be an infected individual with a causal virus of the coronavirus infectious disease (hereinafter, also simply referred to as a “causal virus”) to be subjected to the severity risk evaluation (hereinafter, referred to as a “target coronavirus infectious disease”), or may be a non-infected individual. The term “infected individual” refers to an individual in a state where the causal coronavirus is detectable. The term “non-infected individual” refers to an individual from which no causal coronavirus is detected. Detection of the coronavirus can be performed by a known method (PCR method, antigen method, or the like) using a biological sample collected from a subject (saliva, nasal swab, nasopharyngeal swab, or the like).

In a case where a subject is an individual infected with the causal coronavirus, the subject is preferably at an early stage of infection with the causal coronavirus. When the severity risk is evaluated at an early stage of infection, it is possible to predict whether the subject will develop the target coronavirus infectious disease and the symptom thereof will become severe. As a result, appropriate therapeutic intervention can be performed.

When the severity risk of a non-infected individual, it is possible to predict whether the symptom of the target coronavirus infectious disease will become severe in a case where the subject is infected with the causal virus. As a result, appropriate protective intervention such as vaccination can be performed.

The subject may be a developing individual of the target coronavirus infectious disease or a non-developing individual of the target coronavirus infectious disease. The phrase “developing individual” refers to an individual who has been infected with the causal coronavirus and has developed one or more symptoms of the target coronavirus infectious disease. The phrase “non-developing individual” refers to an individual who has been infected with the causal coronavirus but has developed no symptom of the target coronavirus infectious disease.

In a case where a subject is a developing individual of the target coronavirus infectious disease, the subject is preferably at an early stage of development of the coronavirus infectious disease. When the severity risk is evaluated at an early stage of development, it is possible to predict whether in the subject, the symptom of the target coronavirus infectious disease will become severe. As a result, appropriate therapeutic intervention can be performed.

In a case where a subject is a non-developing individual of the target coronavirus infectious disease and an infected individual with the causal virus, it is possible to predict whether the coronavirus infectious disease will develop and the symptom thereof will become severe in the subject by evaluating the severity risk. As a result, appropriate therapeutic intervention can be performed.

The subject may be an infection-experienced individual of the target coronavirus infectious disease or may be an infection-unexperienced individual. The phrase “infection-experienced individual” refers to an individual who has been infected with the causal virus once or more times and has been cured of the target coronavirus infectious disease. The phrase “infection-unexperienced individual” refers to an individual who has never been infected with the causal virus.

When the severity risk of an infection-experienced individual is evaluated, it is possible to predict whether a subject will develop the target coronavirus infectious disease and the symptom thereof will become severe in a case where the subject is infected with the causal virus again. As a result, appropriate protective intervention such as vaccination can be performed.

When the severity risk of an infection-unexperienced individual is evaluated, it is possible to predict whether a subject will develop the target coronavirus infectious disease and the symptom thereof will become severe in a case where the subject is infected with the causal virus. As a result, appropriate protective intervention such as vaccination can be performed.

The subject may be an individual who has been vaccinated for the target coronavirus infectious disease (vaccinee) or an unvaccinated individual.

When the severity risk of the vaccinee is evaluated, it is possible to evaluate whether the vaccination has succeeded in acquiring sufficient immunity to avoid severe cases of the target coronavirus infectious disease. As a result, appropriate protective intervention such as revaccination can be performed.

When the severity risk of an unvaccinated individual is evaluated, it is possible to evaluate whether the subject has a sufficient immunity to avoid severe cases of the target coronavirus infectious disease without being vaccinated. As a result, appropriate protective intervention such as vaccination can be performed.

### (T Cells of Subject)

T cells of a subject can be isolated from blood of the subject by a known method. For example, a fraction of peripheral blood mononuclear cells (PBMCs) containing T cells can be separated from blood by a density gradient centrifugation method. PBMCs include antigen-presenting cells such as dendritic cells, monocytes/macrophages, and B cells in addition to T-cells (CD8-positive T cells, CD4-positive T cells, and the like). In the step (a), PBMCs may be used as the cell population containing the target T cells.

Alternatively, T cells may be isolated from PBMCs using an antibody or the like that specifically binds to a T-cell marker (CD8, CD4, or the like). In one embodiment, the T cells include at least one selected from the group consisting of CD8-positive T cells and CD4-positive T cells. In one embodiment, the T cells include both CD8-positive T cells and CD4-positive T cells.

(Proteins or Fragments thereof Derived from Another Kind of Coronavirus)

The term “another kind of coronavirus” is a different kind of coronavirus from the causal virus. Another kind of coronavirus (hereinafter also referred to as “antigen virus”) is not particularly limited as long as it is a virus belonging to the family Coronaviridae and is a different kind of coronavirus from the causal virus. In one embodiment, the antigen virus is coronavirus belonging to a different species from the causal virus. In one embodiment, the antigen virus is a coronavirus belonging to the same genus as the causal virus and belonging to a different species from the causal virus. For example, in a case where the causal virus is of the genus Betacoronavirus, the antigen virus may be of a different species of the genus Betacoronavirus. The antigen virus may be of the genus Alphacoronavirus. Preferably, the antigen virus is coronavirus capable of infecting a subject.

For example, in a case where the causal virus is SARS-CoV-2, a virus belonging to the genus Betacoronavirus can be used as the antigen virus. For example, in a case where the causal virus is SARS-CoV-2 and the subject is a human, Human coronavirus (HCoV-OC43, HCoV-HKU1, or the like) belonging to the genus Betacoronavirus can be used as the antigen virus.

The antigen virus may be coronavirus utilizing a different molecule as a receptor from a molecule utilized as a receptor by the causal virus (hereinafter also referred to as “first antigen virus” or “first of another kind of coronavirus”). For example, in a case where the causal virus is SARS-CoV-2 and the subject is a human, coronavirus utilizing a molecule other than ACE2 as a receptor can be used as the antigen virus. Examples of such coronavirus include HCoV-229E, HCoV-OC43, HCoV-HKU1, and MERS-CoV.

The antigen virus may be coronavirus utilizing as a receptor the same molecule as the molecule utilized as a receptor by the causal virus (hereinafter, also referred to as “second antigen virus” or “second of another kind of coronavirus”). For example, in a case where the causal virus is SARS-CoV-2 and the subject is a human, coronavirus utilizing ACE2 as the receptor can be used as the antigen virus. Examples of such coronavirus include HCoV-NL63 and SARS-CoV.

Only one kind of antigen virus may be used, or two or more kinds thereof may be used. As the antigen virus, it is preferable to use both the first antigen virus and the second antigen virus.

As the first antigen virus, one or more selected from the group consisting of HCoV-229E, HCoV-OC43, HCoV-HKU1, and MERS-CoV are preferably used, two or more thereof are more preferably used, and three or more thereof are still more preferably used. As the first antigen virus, all of HCoV-229E, HCoV-OC43, and HCoV-HKU1 are preferably used.

As the second antigen virus, one or more selected from the group consisting of HCoV-NL63 and SARS-CoV are preferably used. As the second antigen virus, HCoV-NL63 is preferably used.

A protein or fragment thereof derived from the antigen virus is used as a T cell stimulating antigen. The phrase “protein derived from the antigen virus” (hereinafter also referred to as “antigen viral protein”) refers to a protein possessed by the antigen virus or a protein expressed by a provirus of the antigen virus. Examples of the antigen viral protein include spike protein (S protein), nucleocapsid protein (N protein), membrane protein (M protein), envelope protein (E protein), and RNA-dependent RNA polymerase (RdRp).

As the antigen viral protein, a protein that binds to a receptor is preferably used. Immune memory against an antigen virus utilizing a receptor the same as that utilized by the causal virus may induce excessive immune response upon infection with the causal virus, resulting in a severe case of the coronavirus infectious disease caused by the causal virus. Coronavirus usually binds to the receptor via the spike protein (S protein). Thus, it is preferable to use the S protein as the antigen viral protein.

The fragment of the antigen viral protein is not particularly limited as long as it has a part of an amino acid sequence of the antigen viral protein. Preferably, the fragment of the antigen viral protein has a size sufficient to activate T cells. For example, a fragment of an antigen viral protein may be a fragment of the antigen viral protein of 8 or more amino acids, 9 or more amino acids, or 10 or more amino acids. In one embodiment, the fragment of the antigen viral protein includes a T cell epitope of the antigen viral protein.

The antigen viral protein may be modified as long as it can induce cellular immunity against the antigen virus. For example, the antigen viral protein may have an amino acid sequence obtained by substitution, deletion, and/or addition of one or several (e.g., 2 to 20, 2 to 15, 2 to 10, 2 to 5, 2 to 4, 2, or 3) amino acids in the amino acid sequence of a protein possessed by a natural antigen virus.

The antigen viral protein or fragment thereof may be synthesized based on the amino acid sequence of the antigen viral protein. Alternatively, it may be synthesized by a cell or cell-free synthesis system using a polynucleotide encoding the antigen viral protein or fragment thereof. The amino acid sequence or gene sequence of the antigen viral protein can be obtained from a sequence database such as GenBank. For example, the amino acid sequence of the S protein of HCoV-OC43 registered under NCBI Reference Sequence: YP_009555241.1 (SEQ ID NO: 1) or GenBank No. AMK59677.1 (SEQ ID NO: 2) can be used. For example, the amino acid sequence of the S protein of HCoV-HKU1 registered under NCBI Reference Sequence: YP_173238.1 (SEQ ID NO: 3) or GenBank No. AYN64561.1 (SEQ ID NO: 4) can be used. For example, the amino acid sequence of the S protein of HCoV-229E registered under NCBI Reference Sequence: NP_073551.1 (SEQ ID NO: 5) can be used. For example, the amino acid sequence of the S protein of HCoV-NL63 registered under NCBI Reference Sequence: YP_003767.1 (SEQ ID NO: 6) can be used.

As the antigen viral protein or fragment thereof, a commercially available product may be used. For example, the S protein of HcoV-OC43 and the S protein of HCoV-HKU1 are commercially available from Sino Biological Inc. and the like. For example, the S protein of SARS-Cov-2 and the peptide pool of SARS-Cov-2 are commercially available from mabtech AB and the like.

The antigen viral protein or fragment thereof is preferably a full-length protein of the antigen viral protein. When the full-length protein is used, the possibility of including a T cell epitope is increased. In addition, it is easy to deal with a mutant strain.

### (Contact Method)

The method for bringing the subject-derived T cell into contact with the antigen viral protein or fragment thereof is not particularly limited. For example, a method commonly used in the ELISPOT assay can be used. In one embodiment, contact between the T cell and the antigen viral protein or fragment thereof is performed in the presence of an antigen-presenting cell. Examples of the antigen-presenting cell include a dendritic cell, a B cell, and a macrophage. The antigen-presenting cell is preferably collected from the subject. PBMCs isolated from blood of the subject include the T cells and the antigen-presenting cells. Thus, PBMCs obtained from the blood of the subject may be used for contact with the antigen viral protein or fragment thereof.

The antigen viral protein or fragment thereof may be presented by the antigen-presenting cell. When the antigen viral protein or fragment thereof is cultured with the antigen-presenting cell, the antigen viral protein or fragment thereof is taken up by the antigen-presenting cell and presented by the antigen-presenting cell as an MHC/peptide complex. The antigen-presenting cell presenting this MHC/peptide complex may be used for contact with the subject-derived T cell.

An amount of the subject-derived T cells used in the reaction is not particularly limited. Examples of the amount of T cells include 10³ to 10⁸ cells, 10⁴ to 10⁷ cells, 10⁴ to 10⁶ cells, and 10³ to 10⁸ cells. In a case where PBMCs are used, examples of the number of PBMCs used in the reaction include from 10³ to 10⁸, from 10⁴ to 10⁷, from 10⁴ to 10⁶, and from 10³ to 10⁸.

An amount of the antigen viral protein or fragment thereof used in the reaction is not particularly limited. Examples of the amount of the antigen viral protein or fragment thereof is 0.1 μg/mL or more, 0.5 μg/mL or more, 1 μg/mL or more, 2 μg/mL or more, 5 μg/mL or more, and 10 μg/mL or more. The upper limit of the antigen viral protein or fragment thereof is not particularly limited, and examples thereof include 100 μg/mL or less, 60 μg/mL or less, 50 μg/mL or less, 40 μg/mL or less, 30 μg/mL or less, 20 μg/mL or less, and 15 μg/mL or less. The upper limit and the lower limit can be arbitrarily combined.

The reaction time is not particularly limited, and only need be a time to the extent that immune response of T cells by the antigen viral protein or fragment thereof is induced. Examples of the reaction time include 1 hour or longer, 2 hours or longer, 5 hours or longer, 7 hours or longer, 10 hours or longer, 15 hours or longer, and 20 hours or longer. The upper limit of the reaction time is not particularly limited and examples thereof include 50 hours or shorter, 40 hours or shorter, 30 hours or shorter, or 25 hours or shorter. The upper limit and the lower limit can be arbitrarily combined.

Examples of the reaction temperature include from 20 to 40° C., from 25 to 40° C., and from 30 to 40° C. The reaction temperature is typically 37° C.

The subject-derived T cell may be brought into contact with the antigen viral protein or fragment thereof by using a well plate for ELISPOT or the like. A commercially available ELISPOT plate can be used. A capture antibody that specifically binds to a cytokine secreted by the T cell upon antigen stimulation may be immobilized on the well plate for ELISPOT. Examples of the cytokine include, but are not limited to, interferon γ (IFN-γ), interleukin 2 (IL-2), IL-4, IL-5, IL-10, IL-13, perforin, granzyme B (GzB), and tumor necrosis factor alpha (TNF-α). One kind or two or more kinds of the capture antibody may be used. In one embodiment, the capture antibody is an anti-IFN-γ antibody and an anti-IL-2 antibody.

The T cell having responsiveness to the antigen viral protein or fragment thereof secretes the cytokine upon contact with the antigen viral protein or fragment thereof. The capture antibody can bind to the cytokine secreted from the T cell and capture it on the well plate.

For immobilizing the capture antibody, the bottom surface of the well of the ELISPOT plate may be formed of a polyvinylidene fluoride (PVDF) membrane.

The subject-derived T cell is preferably brought into contact with the antigen viral protein or fragment thereof for each kind of antigen virus from which the antigen protein or fragment thereof is derived. That is, it is preferable to bring an antigen viral protein derived from one kind of antigen virus or fragment thereof into contact with the T cell. This makes it possible to measure the responsiveness of the T cell for each kind of antigen virus. For example, in a case where four kinds of antigen virus, HCoV-229E, HCoV-OC43, HCoV-HKU1, and HCoV-NL63, are used, contact between a protein or fragment thereof derived from HCoV-229E and the T cell, contact between a protein or fragment thereof derived from HCoV-OC43 or fragments thereof and the T cell, contact between a protein or fragment thereof derived from HCoV-HKU1 and the T cell, and contact between a protein or fragment thereof derived from HCoV-NL63 and the T cell can be performed separately.

As the antigen viral protein or fragment thereof, a plurality of kinds of protein or fragment thereof may be used for one kind of antigen virus, or only one kind of protein or fragment thereof may be used. Preferably, the protein or fragment thereof includes S protein or fragment thereof.

<Step (b)>

In the step (b), the severity risk of the target coronavirus infectious disease is evaluated based on the responsiveness of the subject-derived T cell to the antigen viral protein or fragment thereof.

The responsiveness of the subject-derived T cell to the antigen viral protein or fragment thereof can be measured based on a known method. The T cell proliferates and produces a cytokine when activated by contact with the antigen viral protein or fragment thereof. Thus, the responsiveness of the T cell to the antigen viral protein or fragment thereof may be measured based on T cell proliferation or cytokine production.

For example, the responsiveness of the T cell can be measured based on the cytokine secretion from the T cell against stimulation by the antigen viral protein or fragment thereof. For example, the greater the amount of cytokine secreted from the T cell upon contact with the antigen viral protein or fragment thereof, the higher the responsiveness can be evaluated. The amount of cytokine secretion can be measured by, for example, an immunological technique using an antibody that specifically binds to the cytokine. Examples of such an immunological technique include, but are not limited to, an ELISA method and a Western blotting method. Alternatively, the responsiveness of the T cell may be evaluated based on the amount of mRNA of the cytokine expressed by a T cell population. Examples of the method for measuring the amount of mRNA of the cytokine include, but are not limited to, RT-qPCR and northern blotting.

The responsiveness of the T cell may be evaluated by the number of T cells that secrete a cytokine upon contact with the antigen viral protein or fragment thereof. For example, the greater the number of T cells that secrete the cytokine upon contact with the antigen viral protein or fragment thereof, the higher the responsiveness can be evaluated. The number of T cells secreting the cytokine can be measured by, for example, an immunological technique using an antibody that specifically binds to the cytokine. Examples of such an immunological technique include, but are not limited to, the ELISPOT assay.

For example, the responsiveness of the subject-derived T cell to the antigen viral protein or fragment thereof can be measured using the ELISPOT assay technique. The ELISPOT assay can be performed, for example, as follows. In the ELISPOT assay, T cells and a T cell stimulating antigen (antigen viral protein or fragment thereof) are incubated in a well and a cytokine secreted from the T cells are captured by a capture antibody immobilized in the well. After the well is then washed to remove the T cells, a labeled detection antibody is added to the well. As the detection antibody, an antibody that specifically binds to the same cytokine as the capture antibody can be used. The detection antibody binds to the cytokine captured by the capture antibody. Then, a color development reaction is performed according to the label possessed by the detection antibody, whereby the cytokine can be detected. In the ELISPOT assay, a spot is generated by a chromogenic reaction at a location where the T cell that has secreted the cytokine was positioned. The number of spots can be counted by an ELISPOT reader or the like. When the number of spots is larger, it is possible to evaluate that the responsiveness of the T cell to the antigen viral protein or fragment thereof is high.

The chromogenic reaction for detecting a cytokine can be performed by using an enzyme that catalyzes the chromogenic reaction (hereinafter, referred to as “chromogenic enzyme”) and a substrate for the enzyme. Examples of the chromogenic enzyme include peroxidase (e.g., horseradish peroxidase; HRP) and alkaline phosphatase (AP). The chromogenic enzyme may be directly labeled to the detection antibody, or may be bound to the detection antibody using a binding pair such as biotin-avidin binding. In a case where two or more kinds of cytokines are detected, different chromogenic enzymes may be used for each cytokine.

As the detection antibody, an antibody labeled with a fluorescent pigment may be used. In this case, when the detection antibody is added to the well from which the T cells have been removed and allowed to react, a fluorescent spot is generated at the location where the T cell that had secreted the cytokine was positioned. The number of the fluorescent spots may be counted by a fluorescent spot detection reader or the like.

The responsiveness of the subject-derived T cell to the antigen viral protein or fragment thereof is associated with the severity risk of the target coronavirus infectious disease.

For example, in a case where the responsiveness of the subject-derived T cell to the antigen viral protein or fragment thereof is low, it is possible to evaluate that the subject has a high severity risk of the target coronavirus infectious disease.

For example, in a case where the responsiveness of the subject-derived T cell to the antigen viral protein or fragment thereof is high, it is possible to evaluate that the subject has a low severity risk of the target coronavirus infectious disease.

The high responsiveness of the subject-derived T cell to the antigen viral protein or fragment thereof can mean that the subject has been infected with the antigen virus and cellular immunity against the antigen virus is maintained. It is considered that a T cell having responsiveness to the antigen virus also responds to the causal virus of the target coronavirus infectious disease by cross-reaction. Accordingly, it is considered that in a subject having the cellular immunity against the antigen virus, proliferation of the causal virus is suppressed, which makes it possible to avoid a severe case of the target coronavirus infectious disease.

In a case where the antigen viral protein or fragment thereof is derived from the first antigen virus, it may be evaluated that the severity risk of the target coronavirus infectious disease is high in a case where the responsiveness of the T cell to the antigen protein or fragment thereof is low. The responsiveness to the first antigen virus utilizing a receptor different from that used by the causal virus tends to indicate a negative correlation with the severity risk of the target coronavirus infectious disease. For example, in a case where the causal virus is SARS-CoV-2, examples of the first antigen virus include HCoV-OC43, HCoV-HKU1, HCoV-229E, and MERS-CoV. In a case of using proteins or fragments thereof derived from these coronaviruses as the antigen viral proteins or fragments thereof, it may be evaluated that the severity risk of COVID-19 is high in a case where the responsiveness of the T cell is low.

In a case where the antigen viral protein or fragment thereof is derived from the second antigen virus, it may be evaluated that the severity risk of the target coronavirus infectious disease is high in a case where the responsiveness of the T cell to the antigen protein or fragment thereof is high. The responsiveness to the second antigen virus utilizing the same receptor as that utilized by the causal virus tends to indicate a positive correlation with the severity risk of the target coronavirus infectious disease. For example, in a case where the causal virus is SARS-CoV-2, examples of the second antigen virus include HCoV-NL63 and SARS-CoV. In a case of using proteins or fragments thereof derived from these coronaviruses as the antigen viral proteins or fragments thereof, it may be evaluated that the severity risk of COVID-19 is high in a case where the responsiveness of the T cell is high.

The evaluation of responsiveness of the T cell of the subject may be performed based on a preset reference value. For example, in a case where the responsiveness of the T cell to the antigen viral protein or fragment thereof is higher than the reference value, the responsiveness may be evaluated as high. For example, in a case where the responsiveness of the T cell to the antigen viral protein or fragment thereof is lower than the reference value, the responsiveness may be evaluated as low.

The reference value may be, for example, a numerical value calculated by performing statistical processing or the like on the responsiveness measured in a healthy subject group including an arbitrary number of individuals. Alternatively, it may be a numerical value calculated by performing statistical processing or the like on the responsiveness measured in a group of any number of infected individuals or developing individuals of the target coronavirus infectious disease. The reference value may be, for example, a numerical value calculated from the responsiveness measured in a group of patients in a mild case of the target coronavirus infectious disease. Alternatively, the reference value may be a numerical value calculated from the responsiveness measured in a group of patients in a moderate or severer case of the target coronavirus infectious disease (for example, in a case of COVID-19, Moderate I, Moderate II, and Severe). In a case where the reference value is set from the responsiveness of infected individuals or developing individuals of the target coronavirus infectious disease, it is preferable to use T cells collected from patients in the recovery phase of the target coronavirus infectious disease.

The reference value is preferably set for each kind of antigen virus. For example, in a case where the target coronavirus infectious disease is COVID-19 and HCoV-229E, HCoV-OC43, HCoV-HKU1, and HCoV-NL63 are used as the antigen viruses, a reference value can be set for each of these four kinds of antigen viruses.

In the method according to the present aspect, the step (a) may include the following step (a1) and step (a2):
(a1) a step of bringing the subject-derived T cell into contact with a protein or fragment thereof derived from a first of another kind of coronavirus (first antigen virus) utilizing a different molecule as a receptor from a molecule utilized as a receptor by the causal virus; and
(a2) a step of bringing the subject-derived T cell into contact with a protein or fragment thereof derived from a second of another kind of coronavirus (second antigen virus) utilizing as a receptor the same molecule as the molecule utilized as a receptor by the causal virus.

In the method according to the present aspect, the step (b) may include the following steps (b1) to (b3):
(b1) a step of measuring first responsiveness of the subject-derived T cell to the protein or fragment thereof from the first of another kind of coronavirus (first antigen virus);
(b2) a step of measuring second responsiveness of the subject-derived T cell to the protein or fragment thereof from the second of another kind of coronavirus; and
(b3) a step of evaluating the severity risk of the coronavirus infectious disease based on a ratio value of the second responsiveness to the first responsiveness (second responsiveness/first responsiveness).

In the step (a1), the step (a) only need be performed using the protein or fragment thereof derived from the first antigen virus.

In the step (a2), the step (a) only need be performed using the protein or fragment thereof derived from the second antigen virus.

In the step (b1), the responsiveness of the subject-derived T cell induced in step (a1) only need be measured. The responsiveness obtained in the step (b1) is referred to as first responsiveness.

In the step (b2), the responsiveness of the subject-derived T cell induced in step (a2) only need be measured. The responsiveness obtained in the step (b2) is referred to as second responsiveness.

The responsiveness of T cells can be measured by the method described above. The responsiveness of T cells is preferably measured by the ELISPOT assay. The responsiveness of T cells may be quantified, for example, as the number of effective spots by the ELISPOT assay or as an effective spot number index. The number of effective spots is calculated as a value obtained by subtracting the number of spots in a negative control compartment (well) from the number of spots obtained in a compartment (well) in which T cells have been incubated in presence of the T cell stimulating antigen. The negative control compartment is a compartment in which T cells have been incubated in absence of the T cell stimulating antigen. The effective spot number index is calculated as a value obtained by dividing the number of effective spots by the number of spots in a positive control compartment (well). The positive control compartment is a compartment in which T cells have been incubated using an anti-CD3 antibody to which all T cells strongly react as the T cell stimulating antigen.

In the step (b3), the severity risk of the coronavirus infectious disease is evaluated based on a ratio value of the second responsiveness to the first responsiveness (second responsiveness/first responsiveness). The ratio value (second responsiveness/first responsiveness) may be multiplied by an appropriate coefficient. For example, the severity risk can be evaluated by a severity risk value (Y) obtained by the following equation (1).

$$Y = k ( B / A ) \qquad ( 1 )$$

In the equation (1), Y is the severity risk value; k is a coefficient; A is the first responsiveness; and B is the second responsiveness. The coefficient k can take a numerical value in a range of 1 to 10, for example.

In a case where there are two or more kinds of first antigen viruses, the severity risk value (Y) may be calculated for each of the two or more kinds of first viruses by the equation (1), and the obtained severity risk values may be added up to obtain a comprehensive severity risk value (Y). In a case where the severity risk value (Y) is calculated for each of the two or more kinds of first antigen viruses, an identical value is preferably used as B in the equation (1) for the two or more kinds of first antigen viruses.

For example, the severity risk value (Y) may be calculated by the following equation (2).

$$Y = {k}_{1} ( B / {A}_{1} ) + {k}_{2} ( B / {A}_{2} ) + \qquad ( 2 )$$

In the equation (2), Y is the severity risk value; k₁ and k₂ are coefficients; A₁ is the first responsiveness by the first kind of first antigen virus; A2 is the first responsiveness by the second kind of first antigen virus; and B is the second responsiveness. The coefficients k₁ and k₂ each can take a numerical value in a range of 1 to 10, for example. The same addition may apply to the third and subsequent kinds of first antigen viruses.

In a case where the causal virus is SARS-CoV-2, three species of viruses, HCoV-HKU1, HCoV-OC43, and HCoV-229E, are preferably used as the first antigen viruses. As the second antigen virus, HCoV-NL63 is preferably used. In this case, the severity risk value (Y) may be obtained by the following equation (I).

$$\begin{matrix}[ Math 1 ] & \\ Y = {k}_{1} \frac{( NL 63 )}{( HKU 1 )} + {k}_{2} \frac{( NL 63 )}{( OC 43 )} + {k}_{3} \frac{( NL 63 )}{( 229 E )} & ( I )\end{matrix}$$

In the equation (I), Y is the severity risk value; k₁, k₂, and k₃ are coefficients; NL63 is the responsiveness of the T cell to a protein or fragment thereof derived from HCoV-NL63; HKU1 is the responsiveness of the T cell to a protein or fragment thereof derived from HCoV-HKU1; OC43 is the responsiveness of the T cell to a protein or fragment thereof derived from HCoV-OC43; and 229E is the responsiveness of the T cell to a protein or fragment thereof derived from HCoV-229E. The coefficients k₁, k₂, and k₃ each can take a numerical value in a range of 1 to 10, for example. The coefficients k₁, k₂, and k₃ each may be, for example, a numerical value in a range of 1 to 5, or may be a numerical value in a range of 1 to 3. For example, k₁ is 3, k₂ is 1, and k₃ is 3.

The severity risk value (Y) obtained by the above equation (1), (2), or (I) indicates a positive correlation with the severity risk of the corona infectious disease. Thus, the severity risk of the target corona infectious disease can be evaluated by the severity risk value (Y). For example, in a case where the severity risk value (Y) obtained for a certain subject is higher than a preset reference value (cutoff value), it can be determined that the subject has a high severity risk. In a case where the severity risk value (Y) obtained for a certain subject is lower than the preset reference value (cutoff value), it can be determined that the subject has a low severity risk. The reference value may be, for example, a numerical value obtained by performing statistical processing or the like on the severity risk values (Y) calculated in a group of infected individuals or developing individuals including an arbitrary number of individuals.

According to the method of the present aspect, the severity risk of the coronavirus infectious disease can be evaluated by a simple method. When the severity risk is evaluated, it is possible to take socially and medically appropriate measures.

In the method according to the present aspect, a protein or fragment thereof derived from a different kind of coronavirus (antigen virus) from the causal virus of the target coronavirus infectious disease is used as the T-cell stimulating antigen. Thus, also in an infection-unexperienced individual of the causal virus, and an infection-experienced individual or a vaccinee of the causal virus who has lost cellular immunity to the causal virus, it is possible to evaluate the severity risk of the target coronavirus infectious disease.

According to the method of the present aspect, it is possible to evaluate the severity risk even for an unknown coronavirus infectious disease.

### <Optional Step>

The method according to the present aspect may include an optional step in addition to the steps described above. Examples of the optional step include (c) a step of determining a treatment policy based on the evaluation result of the severity risk of the target coronavirus infectious disease in a case where the subject is an infected individual with the causal virus, (d) a step of performing at least one measure selected from the group consisting of observation of symptoms of the target coronavirus infectious disease and treatment of the target coronavirus infectious disease in a case where the subject evaluated as having a high severity risk of the target coronavirus infectious disease is an infected individual with the causal virus, and (e) a step of performing vaccination of the target coronavirus infectious disease in a case where the subject evaluated as having a high severity risk of the target coronavirus infectious disease is a non-infected individual with the causal virus. In a case where any one of the steps (c) to (e) is included, the method according to the present aspect may be a method for preventing a severe case of the coronavirus infectious disease. In a case where either the step (c) or the step (d) is included, the method according to the present aspect may be a method for treating the coronavirus infectious disease.

### (Step (c))

In a case where the subject is an infected individual with the causal virus, it is possible to determine the treatment policy for the target coronavirus infectious disease based on the severity risk. For an infected individual evaluated as having a low severity risk, it may be determined that only follow-up observation is performed without performing active treatment. For an infected individual evaluated as having a high severity risk, when the progress of the symptom is carefully observed and active treatment is performed from the early stage of infection, there is a possibility that a severe case can be avoided. Thus, for an infected individual evaluated as having a high severity risk, it may be determined to perform active treatment from the early stage of infection. A method for treating the target coronavirus infectious disease can be selected depending on the kind of the target coronavirus. Examples of the treatment method include administration of an antiviral agent, a neutralizing antibody, or the like. In a case where the target coronavirus infectious disease is COVID-19, examples of the antiviral agent include Molnupiravir (trade name: Lagevrio capsule), Nirmatrelvir/Ritonavir (trade name: PAXLOVID Pack), Remdesivir (trade name: VEKLURY intravenous infusion liquid), dexamethasone, tocilizumab (trade name: ACTEMRA), and favipiravir (trade name: Avigan). Examples of the neutralizing antibody include an antibody-cocktail therapy of casirivimab and imdevimab (trade name: Ronapreve injection solution set).

### (Step (d))

In a case where a subject evaluated as having a high severity risk of the target coronavirus infectious disease is an infected individual with the causal virus, when careful follow-up observation is performed from the early stage of infection and active treatment is provided, there is a possibility that a severe case can be avoided. Thus, for an infected individual having a high severity risk, observation of symptoms and/or treatment of the coronavirus infectious disease may be performed. Examples of the treatment method include those described above. In addition, the subject may be admitted to a hospital in preparation for follow-up observation and quick response to a severe case. The step (d) may be a step of performing treatment for the corona infectious disease on an infected individual with the causal virus determined to have a high severity risk. For example, the step (d) may be a step of administering an antiviral agent or a neutralizing antibody against the causal virus to the infected individual with the causal virus determined to have a high severity risk.

### (Step (e))

In a case where a subject evaluated as having a high severity risk of the target coronavirus infectious disease is a non-infected individual with the causal virus, when the subject is infected with the causal virus, there is a severity risk. Thus, such a subject may be vaccinated against the target coronavirus infectious disease. When vaccination is performed to acquire immunity against the target coronavirus infectious disease, the severity risk against the target coronavirus can be reduced in the subject. The step (e) may be a step of performing vaccination of the target coronavirus infectious disease on a non-infected individual determined to have a high severity risk.

### [Kit for Evaluating Severity Risk of Coronavirus Infectious Disease]

A second aspect of the present disclosure is a kit for evaluating a severity risk of a coronavirus infectious disease. The kit according to the present aspect includes a protein (antigen viral protein) or fragment thereof derived from a different kind of coronavirus from the causal virus of the coronavirus infectious disease.

### <Antigen Viral Protein or Fragment Thereof>

Examples of the antigen viral protein or fragment thereof include those described above. The antigen virus is preferably coronavirus belonging to the same genus as the causal virus and belonging to a different species from the causal virus. For example, in a case where the causal virus is of the genus Betacoronavirus, the antigen virus may be a different species of the genus Betacoronavirus. The antigen virus may be of the genus Alphacoronavirus. Preferably, the antigen virus is a coronavirus capable of infecting a subject.

In a case where the causal virus is SARS-CoV-2, a virus belonging to the genus Betacoronavirus can be used as the antigen virus. In a case where the causal virus is SARS-CoV-2 and the subject is a human, Human coronavirus belonging to the genus Betacoronavirus (HCoV-OC43, HCoV-HKU1, etc.) can be used as the antigen virus.

The antigen virus may be a first antigen virus. For example, in a case where the causal virus is SARS-CoV-2 and the subject is a human, an antigen viral protein or fragment thereof derived from HCoV-229E, HCoV-OC43, HCoV-HKU1, or MERS-CoV can be used.

The antigen virus may be a second antigen virus. For example, in a case where the causal virus is SARS-CoV-2 and the subject is a human, an antigen viral protein or fragment thereof derived from HCoV-NL63 or SARS-CoV can be used.

The antigen viral protein or fragment thereof may be of one kind or two or more kinds. Preferably, the antigen viral protein or fragment thereof include both those derived from the first antigen virus and those derived from the second antigen virus. For example, in a case where the causal virus is SARS-CoV-2 and the subject is a human, the kit according to the present aspect preferably includes an antigen viral protein or fragment thereof derived from one or more kinds selected from the group consisting of HCoV-229E, HCoV-OC43, HCoV-HKU1, and MERS-CoV, and an antigen viral protein or fragment thereof derived from one or more kinds selected from the group consisting of HCoV-NL63 and SARS-CoV.

The kit according to the present aspect preferably includes an antigen viral protein or fragment thereof derived from HCoV-HKU1, an antigen viral protein or fragment thereof derived from HCoV-OC43, an antigen viral protein or fragment thereof derived from HCoV-229E, and an antigen viral protein or fragment thereof derived from HCoV-NL63.

Examples of the antigen viral protein include S protein, N protein, M protein, and E protein, and S protein is preferred.

### <Optional Component>

The kit according to the present aspect may include an optional component in addition to the antigen viral protein or fragment thereof. Examples of the optional component include a specific binding substance for a cytokine secreted by a T cell in response to antigen stimulation, a solid-phase carrier, a chromogenic enzyme, a chromogenic enzyme substrate, a washing buffer, a dilution buffer, and an instruction manual.

### (Specific Binding Substance to Cytokine Secreted by T Cell in Response to Antigen Stimulation)

The kit according to the present aspect may include a specific binding substance to a cytokine secreted by a T cell in response to antigen stimulation.

The term “specific binding substance” means a substance having a specific binding property to a specific biomolecule. The phrase “having a specific binding property” means having a high binding affinity for a specific biomolecule but having a very low binding affinity for other biomolecules. The specific binding substance preferably has a high binding property to a particular biomolecule but has almost no binding property to other biomolecules. Examples of the specific binding substance to protein include antibodies and aptamers. In one embodiment, the specific binding substance is an antibody.

Examples of the cytokine secreted by the T cell in response to antigen stimulation include those described above. Specific examples of the specific binding substance to the cytokine include, but are not limited to, anti-IFN-γ antibody, anti-IL-2 antibody, anti-IL-4 antibody, anti-IL-5 antibody, anti-IL-10 antibody, anti-IL-13 antibody, anti-perforin antibody, anti-granzyme B antibody, and anti-TNF-α antibody. One specific binding substance may be used alone, or two or more specific binding substances may be used in combination. In a case of detecting two or more cytokines, a specific binding substance that binds to each of the two or more cytokines is used.

The specific binding substance to the cytokine may be bound to a solid-phase carrier as a capture antibody. Binding of the specific binding substance to the solid-phase carrier can be performed by a known method. Examples of the solid-phase carrier include a well plate. The specific binding substance may be bound to a bottom surface of a well of the well plate. The solid-phase carrier may be, for example, a well plate for ELISPOT. In the solid-phase carrier, the bottom surface of the well may be composed of a PVDF membrane.

The specific binding substance to the cytokine may be labeled with a labeling substance as a detection antibody. The labeling substance may be a chromogenic enzyme or a first element of a binding pair for binding the chromogenic enzyme. Specific examples of the specific binding substance labeled with the first element of the binding pair include a biotin-labeled antibody. Examples of the chromogenic enzyme include those described above. For example, in a case where IFN-γ is detected as the cytokine, specific examples of the specific binding substance include a FITC-labeled anti-human IFN-γ antibody. For example, in a case where IL-2 is detected as the cytokine, specific examples of the specific binding substance include a biotin-labeled anti-human IL-2 antibody.

The detection antibody may be labeled with a fluorescent pigment. As the fluorescent pigment, a known fluorescent pigment can be used without particular limitation. Examples of the fluorescent pigment include, but are not limited to, carboxyfluorescein (FAM), 6-carboxy-4′,5′-dichloro-2′,7′-dimethoxyfluorescein (JOE), fluoresceinisothiocyanate (FITC), tetrachlorofluorescein (TET), 5′-hexachloro-fluorescein-CE phosphoramidite (HEX), phycoerythrin (PE), Cy3, Cy5, Alexa568, and Alexa647.

### (Chromogenic Enzyme)

The chromogenic enzyme is not particularly limited, and a known enzyme can be used. Examples of the chromogenic enzyme include HRP and AP. The chromogenic enzyme may be bound to the detection antibody. In a case where the detection antibody is labeled with the first element of the binding pair (e.g., biotin), the chromogenic enzyme may be labeled with a second element of the binding pair (e.g., avidin or a derivative thereof). For example, In a case where the detection antibody is an FITC-labeled anti-human IFN-γ antibody, the chromogenic enzyme may be FITC-labeled HRP (FITC-HRP). For example, in a case where the detection antibody is a biotin-labeled anti-human IL-2 antibody, the chromogenic enzyme may be streptavidin-labeled AP (Strep-Ap).

### (Chromogenic Enzyme Substrate)

The chromogenic enzyme substrate can be appropriately selected depending on the kind of the chromogenic enzyme.

In a case where the chromogenic enzyme is HRP, examples of the chromogenic enzyme substrate include 3,3′-diaminobenzidine (DAB), 3,3′,5,5′-tetramethylbenzidine (TMB), 2,2′-azinobis [3-ethylbenzothiazoline-6-sulfonic acid](ABTS), and o-phenylenediamine dihydrochloride (OPD).

In a case where the chromogenic enzyme is AP, examples of the chromogenic enzyme substrate include nitro blue tetrazolium chloride (NBT), 5-bromo-4-chloro-3-indolyl phosphate (BCIP), and p-nitrophenyl phosphate (PNPP).

The kit according to the present aspect may be provided as a kit for ELISPOT assay.

The kit according to the present aspect can be used for performing the evaluation method according to the first aspect.

In another aspect, the present disclosure provides a use of an antigen viral protein or fragment thereof for producing a kit for evaluating a severity risk of a coronavirus infectious disease. In another aspect, the present disclosure provides a use of an antigen viral protein or fragment thereof for evaluating a severity risk of a coronavirus infectious disease. In another aspect, the present disclosure provides an antigen viral protein or fragment thereof for evaluating a severity risk of a coronavirus infectious disease. Examples of the antigen viral protein include those described above, and a combination of a protein derived from the first antigen virus and the second antigen viral protein is preferable.

The present disclosure can include the following aspects.

A method for determining a severity risk of a coronavirus infectious disease, the method including: (a) a step of bringing a subject-derived T cell into contact with a protein or fragment thereof derived from another kind of coronavirus which is a different kind of coronavirus from a causal virus of the coronavirus infectious disease, and (b) a step of determining the severity risk of the coronavirus infectious disease based on responsiveness of the T cell to the protein or fragment thereof.

A method for selecting a T cell-containing sample derived from a subject having a high severity risk of a coronavirus infectious disease, the method including: (a) a step of bringing a subject-derived T cell into contact with a protein or fragment thereof derived from another kind of coronavirus which is a different kind of coronavirus from a causal virus of the coronavirus infectious disease, and (b) a step of selecting the T cell-containing sample derived from the subject having a high severity risk of the coronavirus infectious disease based on the responsiveness of the T cell to the proteins or fragment thereof.

### EXAMPLES

Hereinafter, the present invention will be described by the following examples, but the present invention is not limited by the examples.

### Example 1

### <Test Subjects>

Test subjects were COVID-19 convalescent patients treated for COVID-19 at the Tohoku University Hospital and relevant facilities (Kurihara Central Hospital, Kesennuma City Hospital). The test subjects previously received explanation and consented. During the post-healing recovery period, about 10 mL of peripheral blood was collected in a dedicated container. Blood was collected in the same manner from healthy subjects who had not suffered from COVID-19 as a control group.

The severity of COVID-19 in the test subjects was classified as follows according to “Clinical Management of Patients with COVID-19, A guide for front-line healthcare workers, Version 5 (Ministry of Health, Labour and Welfare)” (https://www.mhlw.go.jp/content/000785119.pdf).

Mild: No respiratory symptoms with oxygen saturation (SpO2)≥96%, or coughing only without dyspnea. No finding of pneumonia is obtained in any case.

Moderate I (no respiratory failure): Dyspnea and pneumonia findings are obtained with 93%<SpO2<96%.

Moderate II (with respiratory failure): Oxygen administration is required with SpO2≤93%.

Severe: Admission to ICU or a mechanical ventilator is required.

The test subjects were 3 healthy subjects and 11 COVID-19 infection-experienced subjects (Mild: 4 cases, Moderate I: 3 cases, Moderate II: 4 cases).

### <Preparation of Peripheral Blood Mononuclear Cells (PBMCs)>

Peripheral blood was collected from patients in the healing phase at least 6 weeks after COVID-19 infection and from uninfected subjects as the control group. A BD Vacutainer (trade name) CPT mononuclear cell separation blood collection tube (Becton, Dickinson and Company) was used to perform centrifugation at 1500 to 1800 G at room temperature for 15 minutes, thereby separating and collecting a mononuclear cell layer. Cells were washed with RPMI-1640 supplemented with CTL-wash supplement (Cellular Technologies, Cleveland, OH, USA). Centrifugation was then performed at 300 G at room temperature for 15 minutes. This washing operation was repeated twice. Thereafter, a cell preservation liquid (CELLBANKER2 (Nippon Zenyaku Kogyo Co., Ltd.)) was added thereto and the PBMCs were stored at −80° C.

### <ELISPOT Assay>

Reactivity of T-lymphocyte was evaluated against secreted IFN-γ and IL-2. Human IFN-γ/IL-2 Double-Color ELISPOT Kit: hIFNgIL2-2M, CTL (Cellular Technologies, Cleveland, OH, USA) was used for the ELISPOT assay.

The ELISPOT assay was performed by partially modifying the method described in the literature (Czerkinsky C, et al. Immunol. Methods, 1988.). Specifically, a human IFN-γ/IL-2 capture antibody was coated on a 96-well ELISPOT plate with a PVDF membrane at the bottom surface of the well and incubated overnight at 4° C. After washing the plate with PBS, T-lymphocyte stimulating antigens and a co-stimulatory anti-CD28 antibody were added and incubated for 20 minutes at 37° C. As the T-lymphocyte stimulating antigens, spike (S) protein of the genus Betacoronavirus (HCoV-OC43, HCoV-HKU1), S protein of SARS-CoV-2, and a peptide pool of S protein of SARS-CoV-2+nucleocapsid (N) protein+membrane (M) protein were used. The T lymphocyte stimulating antigens used are indicated in Table 2. No T-lymphocyte stimulating antigen was added to a well of the negative control.

TABLE 2

| T lymphocyte | | Manufacturer | |
|---|---|---|---|
| stimulating antigen | Product name | name | Cat. No. |
| HCoV-OC43 | Human coronavirus | Sino | 40607- |
| S protein | (HCoV-OC43) Spike | Biological | V08B |
| | Protein (S1 + S2 ECD, | | |
| | His Tag) | | |
| HCoV-HKU1 | Human coronavirus | Sino | 40606- |
| S protein | HKU1 (isolate N5) | | |
| | (HCoV-HKU1) | Biological | V08B |
| | Spike Protein (S1 + | | |
| | S2 ECD, His Tag) | | |
| SARS-CoV-2 | SARS-CoV-2 S1 | mabtech | 3629-1 |
| S protein | scanning pool | | |
| SARS-CoV-2 | SARS-CoV-2 SNMO | mabtech | 3622-1 |
| Peptide pool | defined peptide pool | | |

The T lymphocyte stimulating antigen concentration in the above incubation was 2 μg/mL (final concentration) for the S protein of SARS-CoV-2 and the peptide pool. The T lymphocyte stimulating antigen concentration was 12.5 μg/mL (final concentration) for the S protein of the genus β-coronavirus (HCoV-OC43, HCoV-HKU1).

After the above incubation, PBMCs collected from 3 healthy subjects and 11 COVID-19 infection-experienced subjects (Mild: 4 cases, Moderate I: 3 cases, Moderate II: 4 cases) were added at 300000 cells/well. Incubation was then performed at 37° C. for 24 hours. After washing with PBS, washing was performed using 0.05% Tween/PBS. Then, IFN-γ/IL-2 detection antibodies (anti-human IFN gamma (FITC) Detection Ab and anti-human IL-2 (Biotin) Detection Ab) were added and incubation was performed at room temperature for 2 hours. After washing with 0.05% Tween/PBS, a mixed solution of Strep-AP and FITC-HRP was added to the wells and incubation was performed at room temperature for 1 hour. After washing with 0.05% Tween/PBS, washing was performed using distilled water. The chromogenic substrate, Blue developer solution, was then added and incubation was performed at room temperature for 15 minutes. After washing with tap water, washing was performed using distilled water. Red developer solution was then added and incubation was performed at room temperature for 10 minutes. After washing with tap water, natural drying was performed in a clean bench for 24 hours.

The number of reacted T lymphocytes was evaluated as Spot-Forming Cells (SFC) by automatic analysis using an automated immunospot analyzer, CTL (Cellular Technologies, Cleveland, OH, USA). A value obtained by subtracting the number of spots of the negative control group from the counted number of spots (hereinafter also referred to as “effective number of spots”) was evaluated according to the following criteria.

### Evaluation Criteria

3+: 50 or greater
2+: 21 or greater and less than 50
+: 6 or greater and less than 21
±: 4 or greater and less than 6
−: Less than 4

### Results

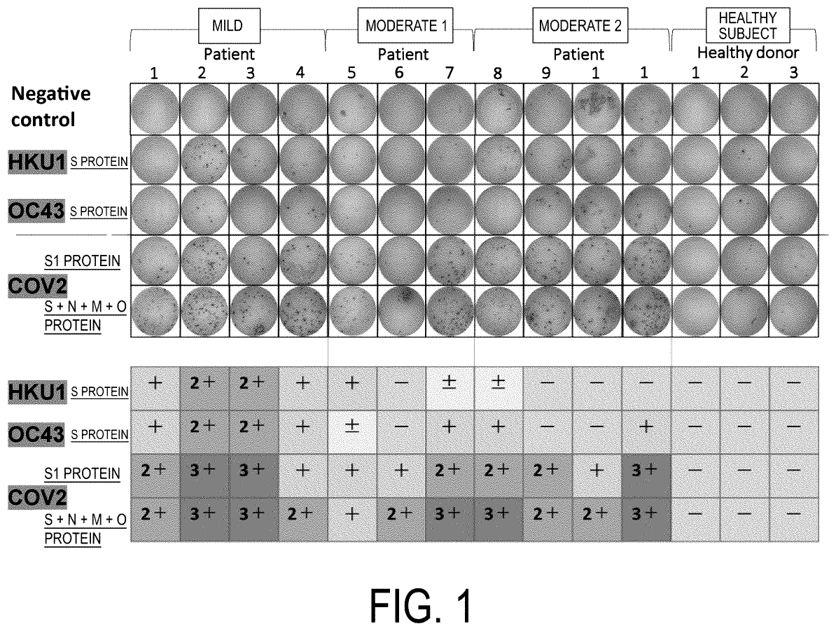

FIG. 1 indicates results of the ELISPOT assay. The upper figure is a well image in the ELISPOT assay. The lower figure indicates evaluation results according to the above evaluation criteria. In the healthy subjects, no response was observed to any of the S proteins of HCoV-OC43, HCoV-HKU1 and SARS-CoV-2.

The COVID convalescent patients indicated moderate to strong responses to HCoV-OC43 and HCoV-HKU1 in the mild cases. In Moderate I, a weak response was observed. In Moderate II, almost no responsiveness was observed.

These results indicate that when there is cell-mediated immunocompetence to the genus Betacoronavirus (HCoV-OC43, HCoV-HKU1) in advance, the cross-reactivity acts in a protective manner against COVID-19 and causes a mild case. On the other hand, it is indicated that the absence of cell-mediated immunocompetence in the genus Betacoronavirus leads to a severe case of COVID-19.

The above results indicated that cross-reactivity in cellular immunity due to the similarity between SARS-CoV-2 and seasonal betacoronavirus reduced the severity of COVID-19.

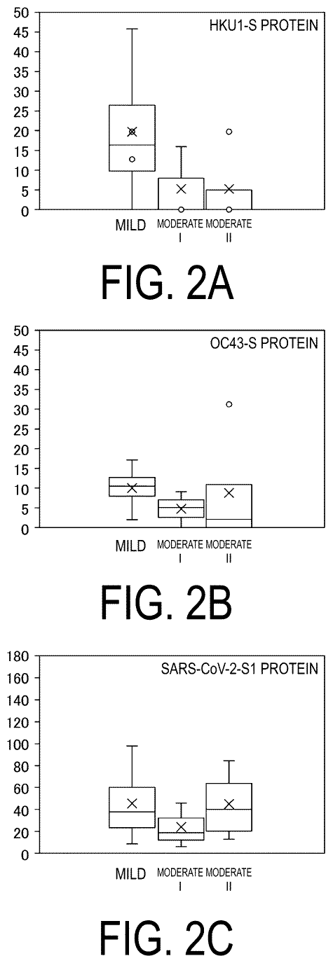

FIGS. 2A to 2C are graphs obtained by quantifying the results of the ELISPOT assay of FIG. 1. In the mild case, it was indicated that the number of effective spots for HCoV-OC43 and HCoV-HKU1 was larger than those for Moderate I and Moderate II. From the result, it has been confirmed that the severity risk of SARS-CoV-2 can be evaluated by the ELISPOT assay using antigen proteins of HCoV-OC43 and/or HCoV-HKU1.

### Example 2

### <Test Subjects>

The test subjects were 37 healthy subjects and 27 COVID-19 infection-experienced subjects (Mild: 8 cases, Moderate I: 6 cases, Moderate II; 9 cases, Severe: 4 cases). As in Example 1, the COVID-19 infection-experienced test subjects were COVID-19 convalescent patients treated for COVID-19 at the Tohoku University Hospital and relevant facilities (Kurihara Central Hospital, Kesennuma City Hospital).

### <Preparation of Peripheral Blood Mononuclear Cells (PBMCs)>

PBMCs were prepared in the same manner as in Example 1 and stored at −80° C.

### <ELISPOT Assay>

The ELISPOT assay was performed using T lymphocyte stimulating antigens indicated in Table 3 in addition to the T lymphocyte stimulating antigens indicated in Table 2.

TABLE 3

| T lymphocyte | | Manufacturer | |
|---|---|---|---|
| stimulating antigen | Product name | name | Cat. No. |
| HCoV-NL63 | Human coronavirus | SinoBio | 40604- |
| S protein | (HCoV-NL63) | | V08B |
| | Spike Protein | | |
| | (S1 + 2 ECD, His-Tag) | | |
| HCoV-229E | Human coronavirus | SinoBio | 40605- |
| S protein | (HCoV-229E) | | V08B |
| | Spike Protein | | |
| | (S1 + 2 ECD, His-Tag) | | |

The ELISPOT assay was performed by a fluorescence method.

Reactivity of T-lymphocyte was evaluated against secreted IFN-γ and IL-2, TNF-α, and GranzymeB. For the ELISPOT assay, Human IFN-γ/IL-2/TNF-α/GranzymeB Four-Color FluoroSPOT Kit: hT4000F-10, CTL (Cellular Technologies, Cleveland, OH, USA) was used. The ELISPOT assay was performed by partially modifying the method described in the literature (Czerkinsky C, et al. Immunol. Methods, 1988.). Specifically, a human IFN-γ/IL-2/TNF-α/GranzymeB capture antibody was coated on a 96-well ELISPOT plate with a PVDF membrane at the bottom surface of the well, and incubated overnight at 4° C. After washing the plate with PBS, T-lymphocyte stimulating antigens and a co-stimulatory anti-CD28 antibody were added and incubated for 20 minutes at 37° C. As the T-lymphocyte stimulating antigens, spike (S) protein of the genus Alphacoronavirus (HCoV-NL63, HCoV-229E) indicated in Table 3 were used in addition to the T-lymphocyte stimulating antigens indicated in Table 2. To a well of the positive control, an anti CD3 antibody was added instead of the T-lymphocyte stimulating antigens. No T-lymphocyte stimulating antigen was added to a well of the negative control.

The T lymphocyte stimulating antigen concentration in the above incubation was 2 μg/mL (final concentration) for the S protein of SARS-CoV-2 and the peptide pool. The T lymphocyte stimulating antigen concentration was 12.5 μg/mL (final concentration) for the S protein of Human cold coronavirus (HCoV-OC43, HCoV-HKU1, HCoV-NL63, HCoV-229E).

After the above incubation, PBMCs collected from the healthy subjects and the COVID-19 infection-experienced subjects were added at 300000 cells/well. Incubation was then performed at 37° C. for 24 hours. After washing with PBS, washing was performed using 0.05% Tween/PBS. Then, IFN-γ/IL-2/TNF-α/GranzymeB detection antibodies (anti-human IFN-γ (FITC) Detection Ab, anti-human IL-2 (Hapten2) Detection Ab, anti-human TNF-α (Hapten1) Detection Ab, and Anti-human GranzymeB (Biotin) Detection Ab) were added and incubation was performed at room temperature for 2 hours. After washing with 0.05% Tween/PBS, a mixed solution of Anti-FITC Alexa Fluor488, Anti-Hapten2 CTL-Red, Anti-Hapten1 CTL-Yellow and Streptavidin eFluor450 was added to the wells and incubation was performed at room temperature for 1 hour. After washing with distilled water, natural drying was performed in a clean bench for 24 hours.

The number of reacted T lymphocytes was evaluated as Spot-Forming Cells (SFC) by automatic analysis using an automated immunospot analyzer, CTL (Cellular Technologies, Cleveland, OH, USA).

### <Results of ELISPOT Assay>

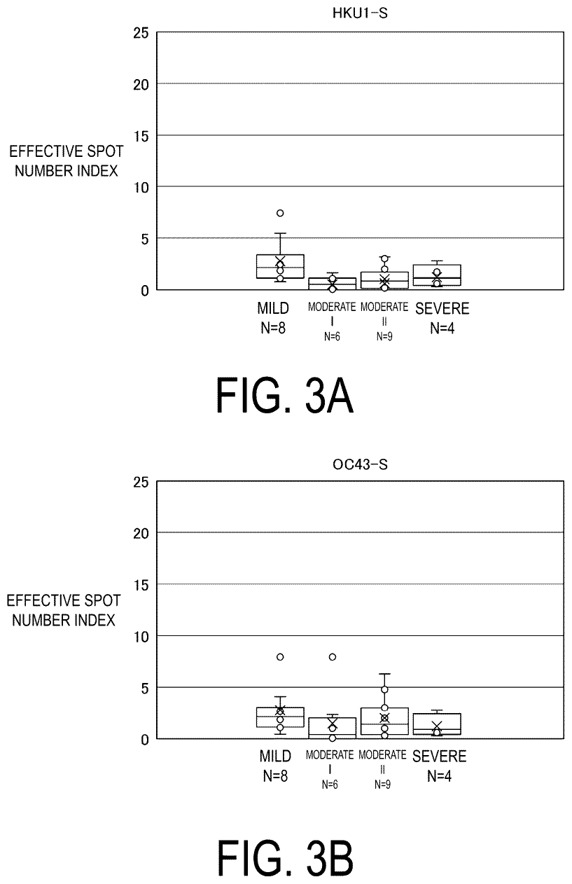

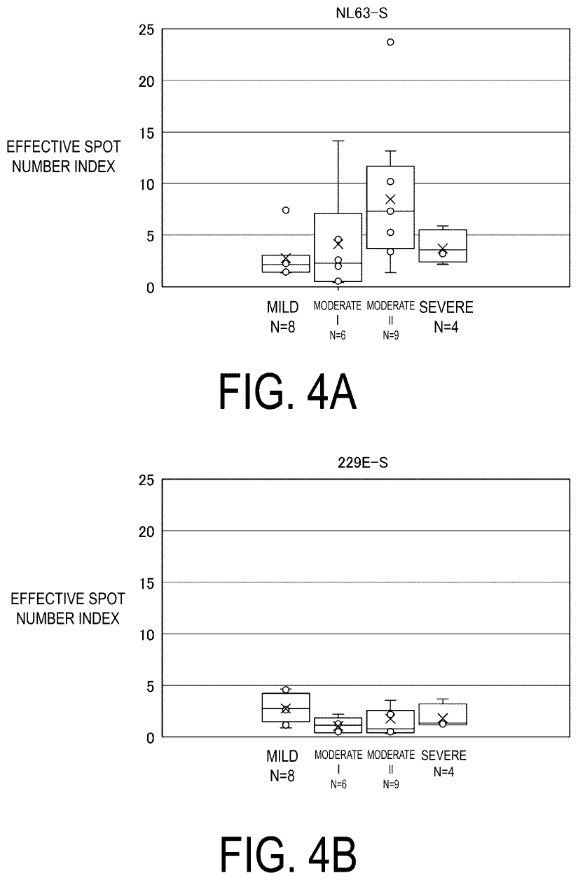

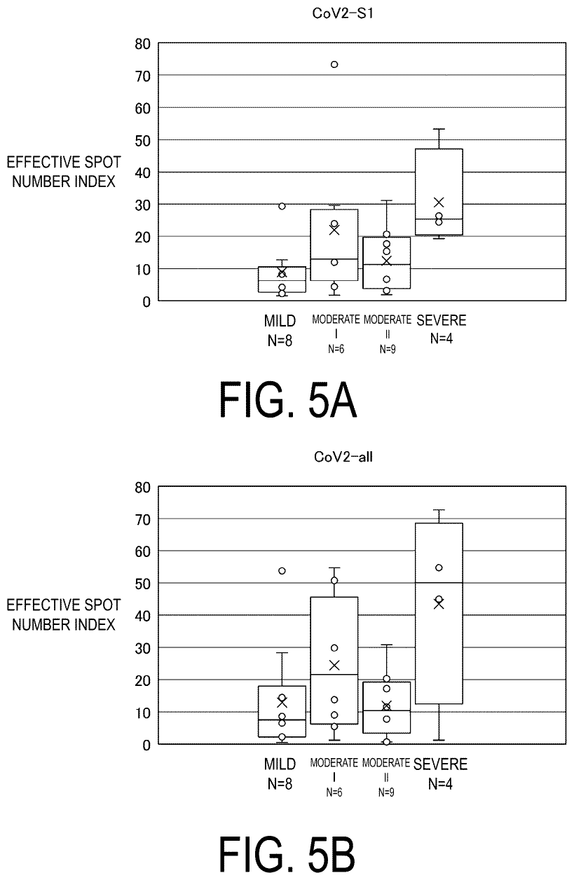

Results of the ELISPOT assay using, as the T lymphocyte stimulating antigens, the S protein of HCoV-HKU1 (HKU1-S) (FIG. 3A), the S protein of HCoV-OC43 (OC43-S) (FIG. 3B), the S protein of HCoV-NL63 (NL63-S) (FIG. 4A), the S protein of HCoV-229E (229E-S) (FIG. 4B), the S protein of SARS-CoV-2 (CoV2-S) (FIG. 5A), and the peptide pool of SARS-CoV-2 (CoV2-all) (FIG. 5B) are indicated in FIG. 3A to FIG. 5B, respectively.

For the S protein of coronaviruses (HKU1 and OC43) belonging to the genus Betacoronavirus and not using ACE2 as a receptor, the reaction tended to be strong in the mild cases, as compared with the moderate or severer cases (FIG. 3A, FIG. 3B).

For the S protein of coronavirus (NL63) belonging to the genus Alphacoronavirus and utilizing ACE2 as a receptor, the reaction tended to be strong in the moderate or severer cases, as compared with the mild cases (FIG. 4A).

For the S protein of coronavirus (229E) belonging to the genus Alphacoronavirus and not utilizing ACE2 as a receptor, the reaction tended to be strong in the mild cases, as compared with the moderate or severer cases (FIG. 4A).

For the proteins of SARS-CoV-2, the reaction tended to be stronger when the severity was higher (FIG. 5A, FIG. 5B).

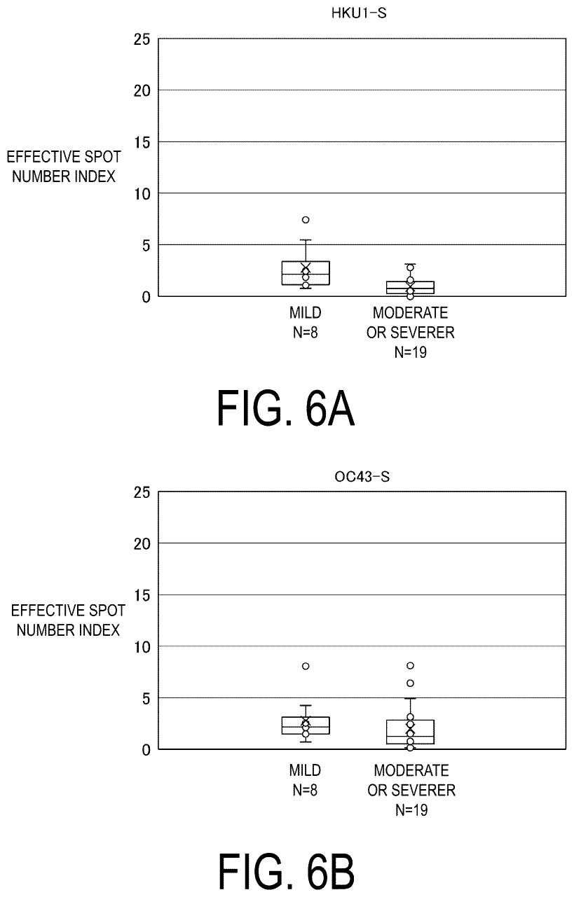

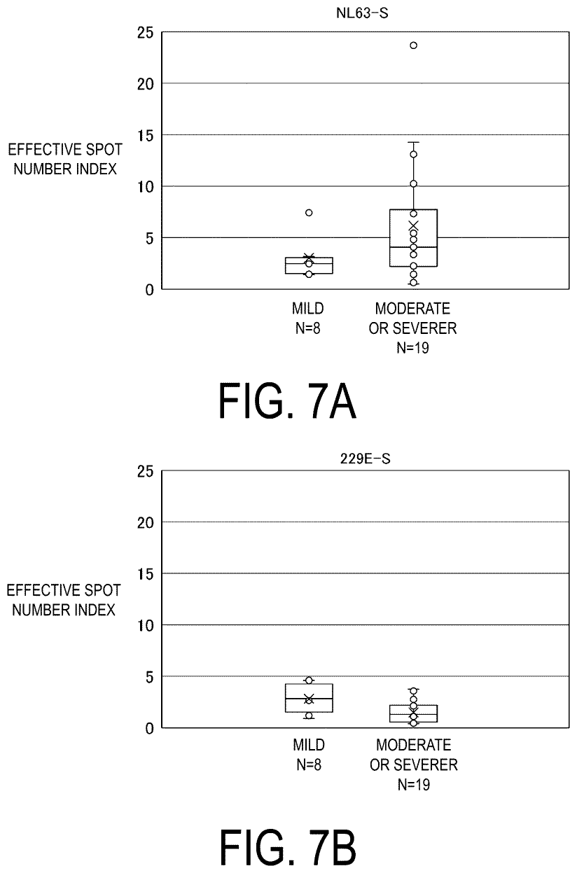

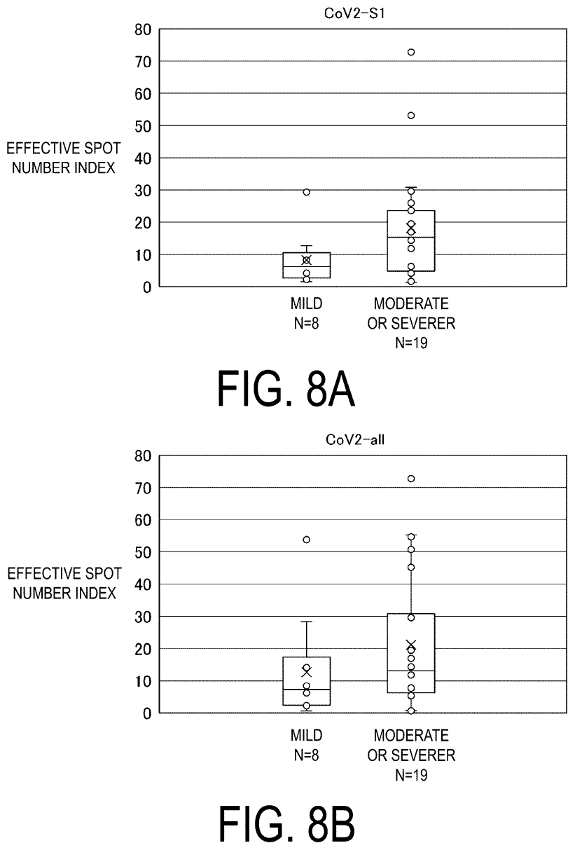

FIG. 6A to FIG. 8B indicate results in a case of summarizing Moderate I, Moderate II, and Severe as “moderate or severer”. In any case, the above-described tendency became more remarkable.

### <Examination of Calculation Equation of Severity Risk Value>

From the above results, the following tendency was confirmed.

(a) For the coronavirus (NL63) utilizing ACE2 as a receptor, the reaction is stronger in the moderate or severer cases than in the mild cases.

(b) For the coronavirus (HKU1, OC43, 229E) not utilizing ACE2 as a receptor, the reaction is stronger in the mild cases than in the moderate or severer cases.

In consideration of the above results, a calculation equation capable of calculating a predicted value of the COVID-19 severity risk was examined from the effective spot number index of the ELISPOT assay. As a result, the following equation (I) was created as an equation for calculating the severity risk value (Y).

$$\begin{matrix}[ Math 2 ] & \\ Y = {k}_{1} \frac{( NL 63 )}{( HKU 1 )} + {k}_{2} \frac{( NL 63 )}{( OC 43 )} + {k}_{3} \frac{( NL 63 )}{( 229 E )} & ( I )\end{matrix}$$

In the equation (I), NL63, HKU1, OC43, and 229E are effective spot number indexes when the ELISPOT assay was performed using NL63-S, HKU1-S, OC43-S, and 229E-S as the T lymphocyte stimulating antigens, respectively.

In the equation (I), k₁, k₂, and k₃ are coefficients.

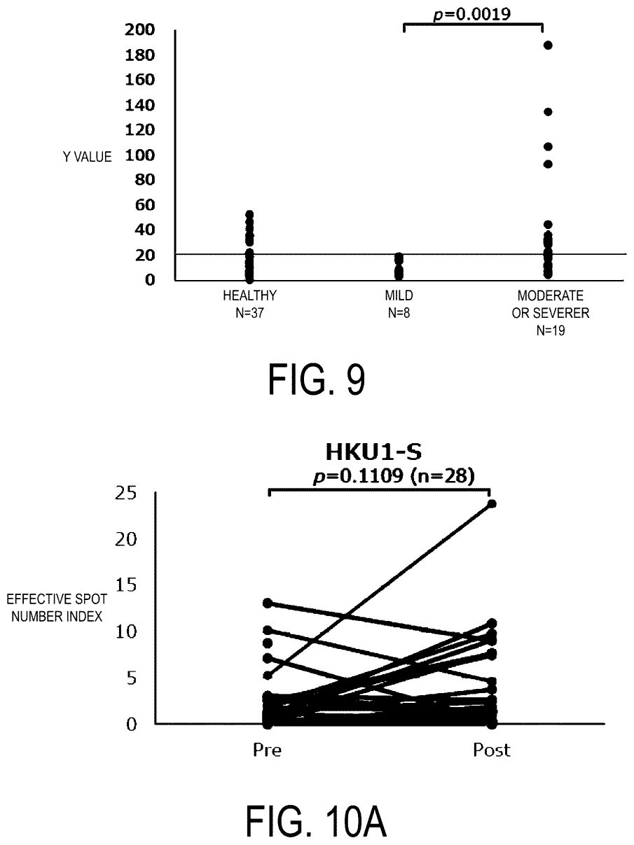

FIG. 9 is a graph illustrating Y values obtained by the equation (1) in groups of healthy subjects, mild cases, and moderate or severer cases. In FIG. 9, k₁=3, k₂=1, and k₃=3 are used as the coefficients in the equation (I). As indicated in FIG. 9, the Y values were significantly higher in the moderate or severer cases than in the mild cases.

In a case where a cut-off value (X) of the Y values was set to 20, the following results were obtained.

X>20: Moderate or severer with a probability of 100%.

X≤20: Mild with a probability of 8/15 (53%).

From the above results, it was confirmed that the severity risk of COVID-19 can be determined by the above equation (I).

### Example 3

### <Test Subjects>

The test subjects were vaccinees vaccinated with vaccine available from Pfizer (BNT162b2). The number of test subjects was 28. Blood was collected before the vaccination and after the vaccination (approximately 6 to 9 weeks later).

### <Preparation of Peripheral Blood Mononuclear Cells (PBMCs), ELISPOT Assay>

Preparation of PBMCs and the ELISPOT assay were performed in the same manner as in Example 2.

### <Results of ELISPOT Assay>

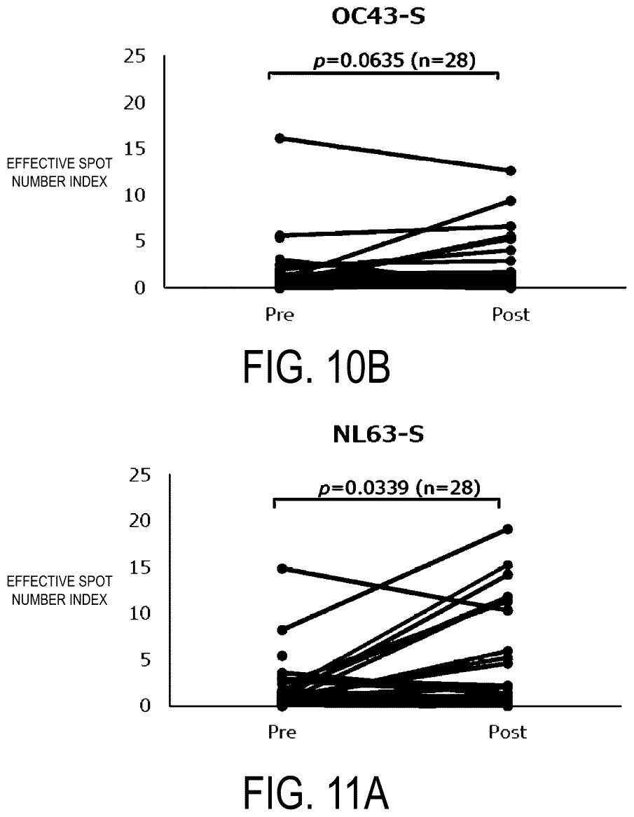

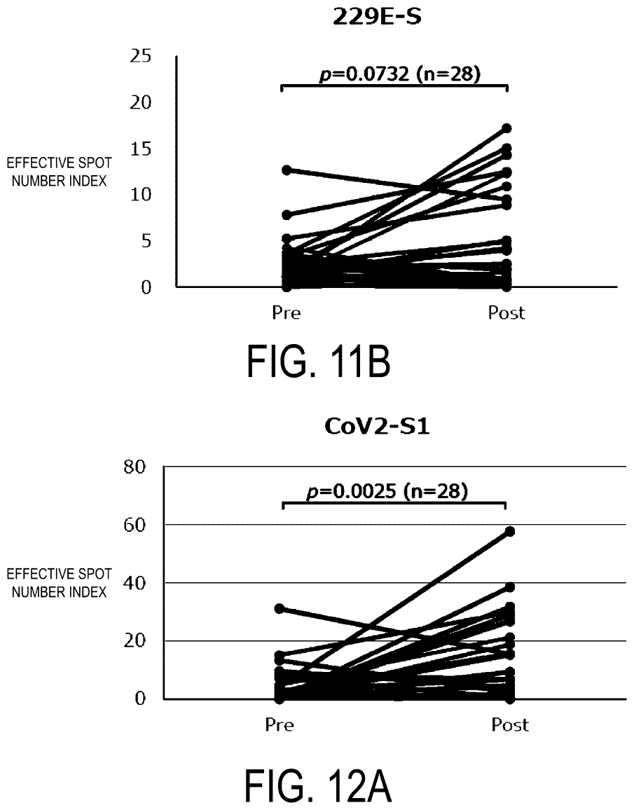

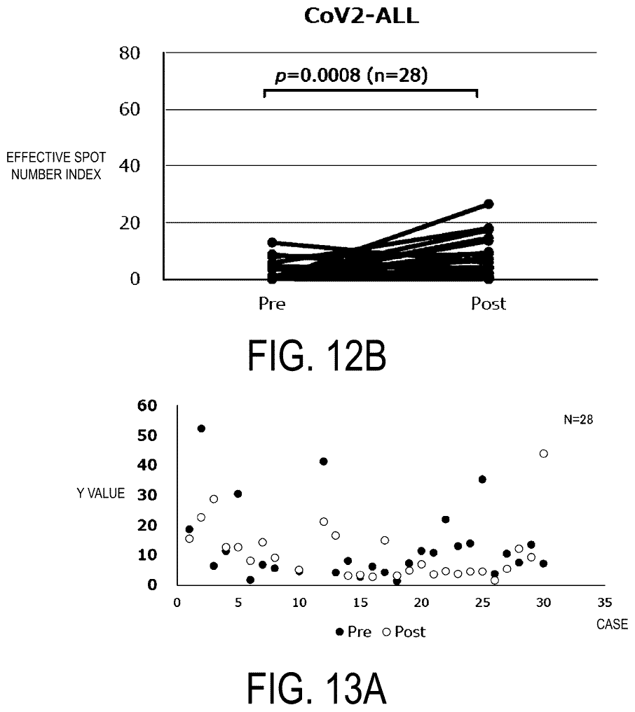

Results of the ELISPOT assay using, as the T lymphocyte stimulating antigens, HKU1-S (FIG. 10A), OC43-S (FIG. 10B), NL63-S (FIG. 11A), 229E-S (FIG. 11B), CoV2-S (FIG. 12A), and CoV2-all (FIG. 12B) are indicated in FIG. 10A to FIG. 12B, respectively. In the figures, “Pre” indicates a result before the vaccination, and “Post” indicates a result after the vaccination.

There was no significant difference in response to the S protein of the coronavirus (HKU1, OC43, 229E) not utilizing ACE2 as a receptor between before and after the vaccination (FIG. 10A, FIG. 10B, FIG. 11B).

The response to the S protein of the coronavirus (NL63) utilizing ACE2 as a receptor was significantly increased after the vaccination, as compared with before the vaccination (FIG. 11A).

The response to the SARS-CoV-2 protein was significantly increased after the vaccination, as compared with before the vaccination (FIG. 12A, FIG. 12B).

### <Severity Risk Value (Y) Before and After Vaccination>

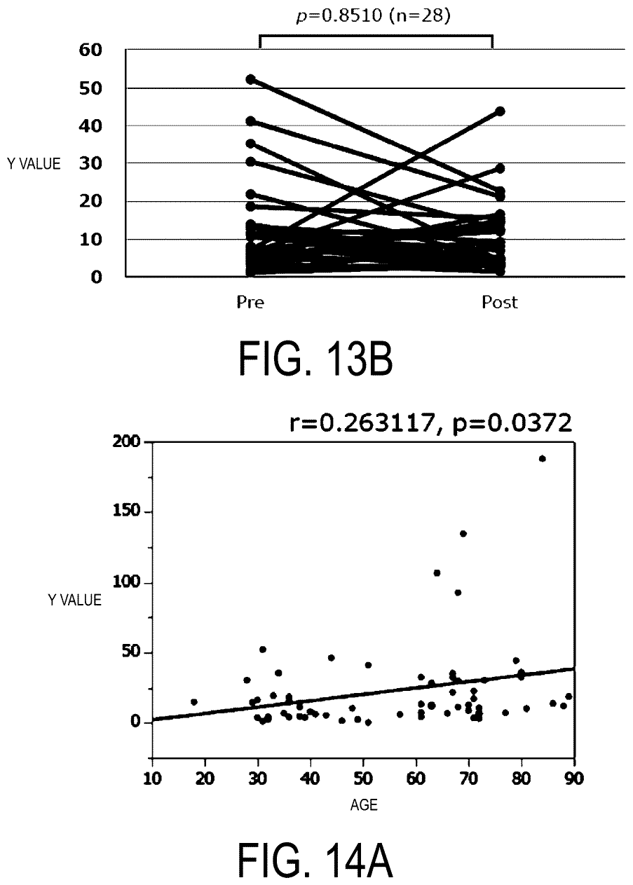

The severity risk values (Y) were calculated by the above equation (I) before and after the vaccination. The results are indicated in FIG. 13A and FIG. 13B.

There was no significant difference in the Y values before and after the vaccination. However, in some test subjects, the Y values clearly decreased after the vaccination (FIG. 13B).

### Example 4

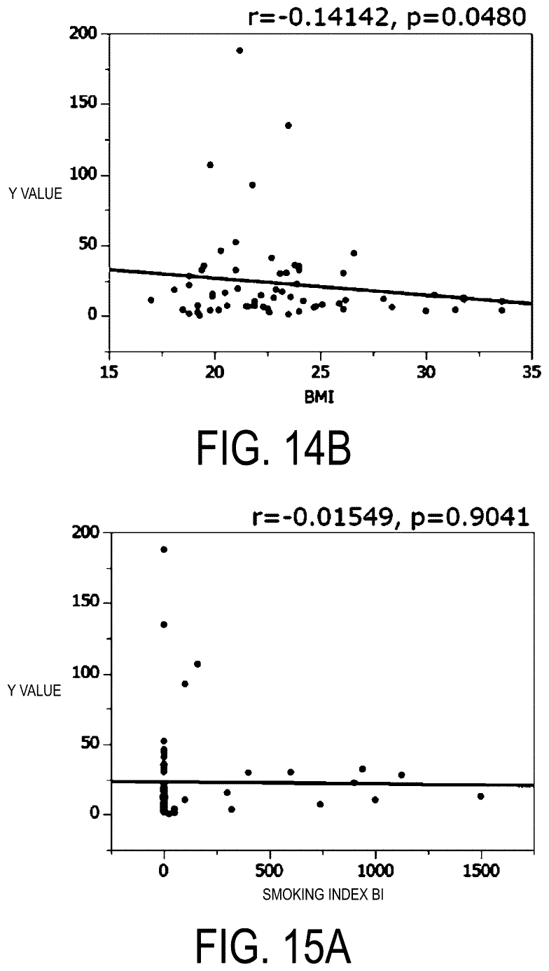

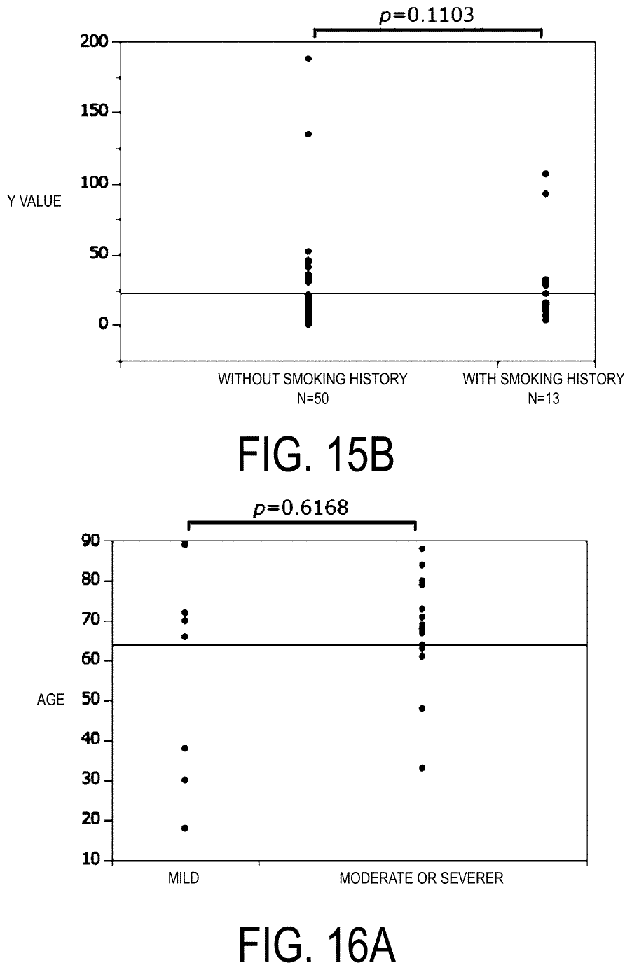

FIG. 14A to FIG. 15B indicate results of analyzing a correlation between a known COVID-19 severity risk factor and the severity risk value (Y). The severity risk value (Y) did not have a significant correlation with any of age (FIG. 14A), BMI (FIG. 14B), smoking index BI (FIG. 15A), and smoking history (FIG. 15B).

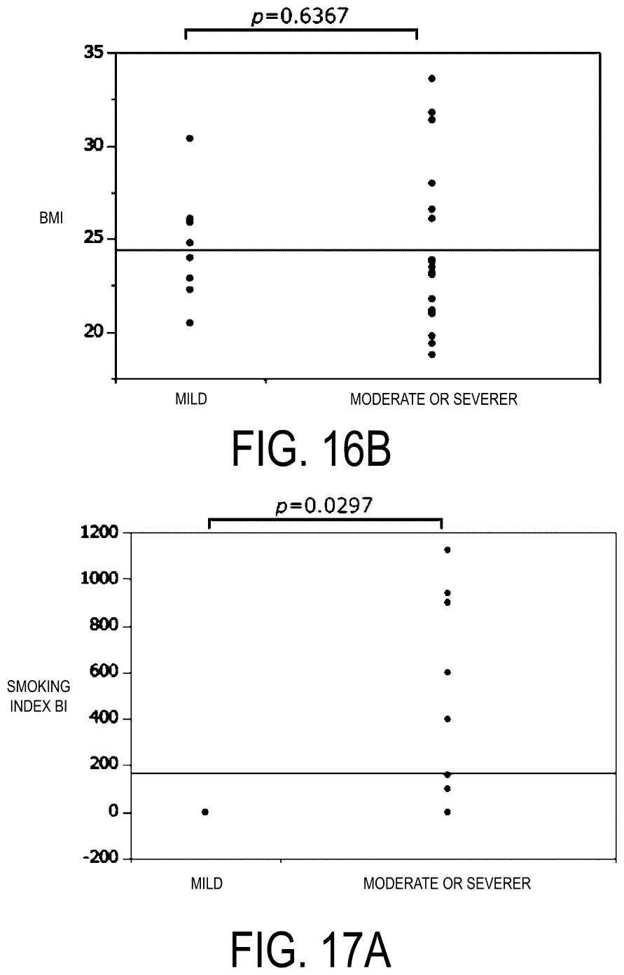

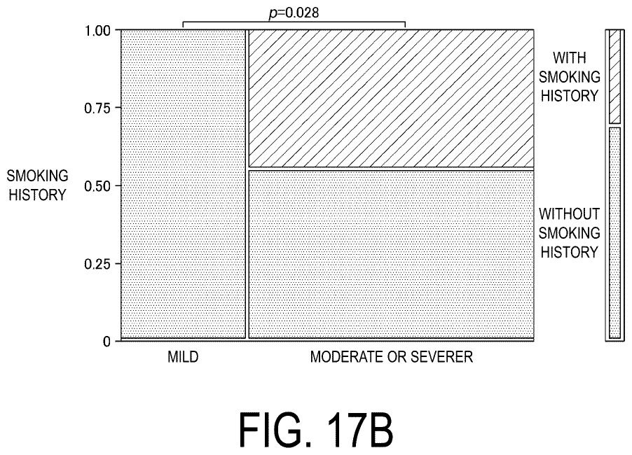

In the test subjects of Example 2, the severity did not have a significant correlation with any of age (FIG. 16A) and BMI (FIG. 16B). In the test subjects of Example 2, the severity had a significant correlation with smoking index BI (FIG. 17A) and smoking history (FIG. 17B).

### INDUSTRIAL APPLICABILITY

According to the present invention, there are provided a method for evaluating a severity risk of a coronavirus infectious disease and a kit for evaluating a severity risk of a coronavirus infectious disease, which can easily determine a severity risk of a coronavirus infectious disease. The evaluation method and the evaluation kit according to the present invention can be applied to the evaluation of the severity risk of COVID-19. They can also be applied to a future unknown coronavirus infectious disease.

Although the preferred embodiments of the present invention have been described above and illustrated, it is to be understood that they are illustrative of the present invention and are not to be considered as limitation. Addition, omission, substitution, and other changes may be made without departing from the spirit or scope of the present invention. Accordingly, the present invention is not to be seen as limited by the foregoing description, but is only limited by the scope of the appended claims.

1.-13. (canceled)

14. A method for evaluating a severity risk of a coronavirus infectious disease, the method comprising:
(a) a step of bringing a subject-derived T cell into contact with a protein or fragment thereof derived from another kind of coronavirus which is a different kind of coronavirus from a causal virus of the coronavirus infectious disease; and
(b) a step of evaluating a severity risk of the coronavirus infectious disease based on responsiveness of the T cell to the protein or fragment thereof.

15. The method for evaluating a severity risk of a coronavirus infectious disease according to claim 14, wherein
the protein or fragment thereof is derived from a first of another kind of coronavirus utilizing a different molecule as a receptor from a molecule utilized as a receptor by the causal virus, and
the step (b) is a step of evaluating that the severity risk of the coronavirus infectious disease is high in a case where the responsiveness of the T cell to the protein or fragment thereof is low.

16. The method for evaluating a severity risk of a coronavirus infectious disease according to claim 14, wherein
the protein or fragment thereof is derived from a second of another kind of coronavirus utilizing as a receptor the same molecule as the molecule utilized as a receptor by the causal virus, and
the step (b) is a step of evaluating that the severity risk of the coronavirus infectious disease is high in a case where the responsiveness of the T cell to the protein or fragment thereof is high.

17. The method for evaluating a severity risk of a coronavirus infectious disease according to claim 14, wherein the step (a) comprises:
(a1) a step of bringing the T cell into contact with a protein or fragment thereof derived from a first of another kind of coronavirus utilizing a different molecule as a receptor from a molecule utilized as a receptor by the causal virus; and
(a2) a step of bringing the T cell into contact with a protein or fragment thereof derived from a second of another kind of coronavirus utilizing as a receptor the same molecule as the molecule utilized as a receptor by the causal virus, and
the step (b) comprises:
(b1) a step of measuring a first responsiveness of the T cell to the protein or fragment thereof derived from the first of another kind of coronavirus;
(b2) a step of measuring a second responsiveness of the T cell to the protein or fragment thereof derived from the second of another kind of coronavirus; and
(b3) a step of evaluating a severity risk of the coronavirus infectious disease based on a ratio value of the second responsiveness to the first responsiveness (second responsiveness/first responsiveness).

18. The method for evaluating a severity risk of a coronavirus infectious disease according to claim 14, wherein the coronavirus from which the causal virus and the protein or fragment thereof are derived are of the genus Betacoronavirus or the genus Alphacoronavirus.

19. The method for evaluating a severity risk of a coronavirus infectious disease according to claim 14, wherein the causal virus is SARS-CoV-2.

20. A kit for evaluating a severity risk of a coronavirus infectious disease, the kit comprising
a protein or fragment thereof derived from another kind of coronavirus which is a different kind of coronavirus from a causal virus of the coronavirus infectious disease.

21. The kit for evaluating a severity risk of a coronavirus infectious disease according to claim 20, the kit further comprising a specific binding substance for a cytokine secreted by a T cell in response to antigen stimulation.

22. The kit for evaluating a severity risk of a coronavirus infectious disease according to claim 21, the kit comprising: the specific binding substance bound to a solid-phase carrier; and the specific binding substance labeled with a labeling substance.

23. The kit for evaluating a severity risk of a coronavirus infectious disease according to claim 20, wherein the cytokine is at least one selected from the group consisting of interferon γ, interleukin 2, and tumor-necrosis factor alpha.

24. The kit for evaluating a severity risk of a coronavirus infectious disease according to claim 20, wherein the protein or fragment thereof comprises:
a protein or fragment thereof derived from a first of another kind of coronavirus utilizing a different molecule as a receptor from a molecule utilized as a receptor by the causal virus; and
a protein or fragment thereof derived from a second of another kind of coronavirus utilizing as a receptor the same molecule as the molecule utilized as a receptor by the causal virus.

25. The kit for evaluating a severity risk of a coronavirus infectious disease according to claim 20, wherein the causal virus and the coronavirus from which the protein or fragment thereof is derived are of the genus Betacoronavirus or the genus Alphacoronavirus.

26. The kit for evaluating a severity risk of a coronavirus infectious disease according to claim 20, wherein the causal virus is SARS-CoV-2.
